(19) United States
(12) Patent Application Publication
(10) Pub. No.: US 2017/0096549 A1
(43) Pub. Date: Apr. 6, 2017

(54) MULTIVALENT CATION FORMULATIONS OF PARTIALLY DESULFATED HEPARINS

(71) Applicant: Cantex Pharmaceuticals, Inc., Weston, FL (US)

(72) Inventors: Gregory JOHNSON, Gainesville, GA (US); Stephen MARCUS, Weston, FL (US)

(73) Assignee: Cantex Pharmaceuticals, Inc., Weston, FL (US)

(21) Appl. No.: 15/126,289

(22) PCT Filed: Mar. 17, 2015
(86) PCT No.: PCT/US15/21068

§ 371 Date: Sep. 15, 2016

Related U.S. Application Data
(60) Provisional application No. 61/954,374, filed on Mar. 17, 2014.

(51) Int. Cl. C08L 5/10; A61K 31/727; A61K 47/02

(52) U.S. Cl. CPC C08L 5/10; A61K 47/02; A61K 31/727

(57) ABSTRACT

Multivalent cation compositions of partially desulfated heparins are presented. Multivalent cation compositions of the disclosure can be formulated at high concentrations. The compositions, processes for making the compositions, unit dosage forms, kits, and methods of treatment are also disclosed.

### 1. CROSS-REFERENCE TO RELATED APPLICATIONS

This application claims the benefit under 35 U.S.C. §119(e) of U.S. Provisional Application No. 61/954,374, filed Mar. 17, 2014, the entire contents of which is incorporated herein by reference.

### 2. BACKGROUND

Heparins are a class of highly anionic glycosaminoglycan (GAG) molecules. Unfractionated heparin (UFH) has substantial anti-coagulant activity and is widely used for that purpose. Heparins also have other biological activities, but UFH and other anti-coagulating heparins typically cannot be administered in amounts sufficient for these additional activities to provide therapeutic benefit, due to excessive anticoagulation.

It has previously been shown that desulfation of the 2-O and/or 3-O positions results in a heparin derivative that has substantially reduced anti-coagulant activity but that retains other activities. See, e.g., Rao et al., 299:C97-C110 (2010). Such 2-O, 3-O-desulfated heparin derivatives, such as the substantially 2-O, 3-O-desulfated heparin derivative, ODSH, can be administered to patients in amounts sufficient to permit these additional activities to provide therapeutic benefit. See, e.g., U.S. Pat. Nos. 5,668,118, 5,707,974, 5,990,097, 6,489,311; U.S. pre-grant publication nos. 2009/0054374, 2009/0036405, 2012/0196828, 2013/0303481; international patent publication no. WO 01/19376.

Current clinical trials of 2-O, 3-O-desulfated heparins, such as that described in US 2013/0303481, require intravenous administration. Although other routes of administration, such as administration by inhalation and administration by subcutaneous injection, have been described, see, e.g. U.S. Pat. No. 5,668,118, WO 98/004133, WO 01/19376, US 2009/0054374, US 2009/0036405, current preparations of 2-O, 3-O-desulfated heparins, such as ODSH, are too dilute to make such non-intravenous routes of administration readily feasible.

There is, therefore, a need in the art for a formulation of substantially non-anticoagulating heparins, such as 2-O, 3-O-desulfated heparins, having sufficiently high concentration as to permit administration by routes other than intravenous administration at dosages that provide clinical benefit.

### 3. SUMMARY

In a first aspect, pharmaceutical compositions comprising a partially desulfated heparin, a multivalent cation, and a pharmaceutically acceptable carrier are provided. In certain embodiments, the partially desulfated heparin is desulfated at the 2-O and/or 3-O positions. In certain embodiments, the multivalent cation is a divalent cation.

A second aspect of the present disclosure is a process for making desulfated heparins with multivalent cations. In one embodiment, the multivalent cation is present during the desulfation of heparin. In one embodiment, the multivalent cation is added after the desulfation step. A third aspect of the present disclosure are the products formed by the processes disclosed herein.

Several aspects of the disclosure are unit dosage forms comprising the pharmaceutical compositions or products of the present disclosure. In one aspect, the unit dosage form is a preloaded syringe. In another aspect, the unit dosage form is an autoinject device. In another aspect, the unit dosage form is an autoinject pen. In another aspect, the unit dosage form is a metered dose inhaler. In some aspects, the unit dosage form is a vial or ampule. The unit dosage forms may be in forms suitable for specific applications or specific devices. In some aspects, the unit dosage form is suitable for use with a nebulizer or aerosolizer.

In another aspect, the unit dosage forms are part of kits. The kits may contain instructions for use of the unit dosage form. The kits may contain other therapies for use with the unit dosage form containing the pharmaceutical composition. The kit may also contain other items useful for the purposes of the kit.

Another aspect of the present disclosure are methods for treating or preventing conditions that are treatable or preventable with a partially desulfated heparin. Partially desulfated heparins have been shown to be useful many disorders, diseases and conditions.

The partially desulfated heparins of the present disclosure can be tailored for particular uses. As will be discussed further, the level of desulfation and identity of the cation can each be matched to a particular use. Solutions of the present disclosure can be made at several concentrations that retain the ability to be safely and comfortably administered to a subject.

### 4. DETAILED DESCRIPTION

### 4.1. Pharmaceutical Compositions

In a first aspect, pharmaceutical compositions are provided that comprise a partially desulfated heparin, a multivalent cation, and a pharmaceutically acceptable carrier.

4.1.1. Partially Desulfated Heparins

Partially desulfated heparins used in the pharmaceutical compositions described herein are prepared from heparins. Heparins are a class of linear glycosaminoglycan (GAG) molecules made up of alternating or repeating amino and uronic saccharide residues. Typical saccharide residues are iduronic acid and glucosamine. As used herein, “heparins” refer to the class of molecules including native heparin, unfractionated heparin (UFH), as well as chemical and biological derivatives, substitutions and modifications thereof. The saccharide residues can have N-sulfate, and/or N-acetyl, and/or O-sulfate substitutions. Common O-sulfate substitutions are 2-O sulfate, 3-O sulfate and 6-O sulfate. Partially desulfated heparins used in the pharmaceutical compositions described herein may be made from heparins having these and other substitutions and modifications, which may be natural or synthetic.

In typical embodiments, the partially-desulfated heparins are partially desulfated at the 2-O position of α-L-iduronic acid (referred to herein as the “2-O position”) and/or partially desulfated at the 3-O position of D-glucosamine-N-sulfate (6-sulfate) (referred to herein as the “3-O position”). In some embodiments, the partially-desulfated heparins are at least 50%, at least 80%, at least 85%, at least 90%, at least 95%, at least 96%, at least 97%, at least 98%, or at least 99% desulfated at the 2-O position. In selected embodiments, the heparins are at least 99% desulfated at the 2-O position. In some embodiments, the partially desulfated heparins are at least 50%, at least 80%, at least 85%, at least 90%, at least 95%, at least 96%, at least 97%, at least 98%, or at least 99% desulfated at the 3-O position. In selected embodiments, the partially desulfated heparins are at least 99% desulfated at the 3-O position. In some embodiments, the partially desulfated heparins are at least 50%, at least 80%, at least 85%, at least 90%, at least 95%, at least 96%, at least 97%, at least 98%, or at least 99% desulfated at both the 2-O position and the 3-O position. In selected embodiments, the partially-desulfated heparins are at least 99% desulfated at the 2-O position and the 3-O position.

In various embodiments, the partially-desulfated heparins are at least 90% desulfated at each of the 2-O and 3-O positions. These embodiments are said to be “substantially desulfated”.

In typical embodiments, the partially desulfated heparins comprise substantially N-sulfated and 6-O sulfated D-glucosamine In some embodiments, the carboxylates on α-L-iduronic acid sugars of partially desulfated heparin are substantially intact.

Heparins of any size can be desulfated to prepare the partially desulfated heparins used in the pharmaceutical compositions described herein. Molecular weights of heparins can be determined by high performance size exclusion chromatography as is known in the art. See, e.g., Lapierre et al., Glycobiology 6(3):355-366 (1996), at page 363; Fryer et al., J. Pharmacol. Exp. Ther. 282:208-219 (1997), at page 209.

Accordingly, the partially-desulfated heparins used in the pharmaceutical compositions can likewise be of any molecular weight. In typical embodiments, the partially-desulfated heparins will have an average molecular weight of about 2-25 kDa. In various embodiments, the average molecular weight will be about 8-15 kDa. In certain embodiments, the average molecular weight will be about 11-13 kDa.

In some embodiments, the average molecular weight is greater than about 0.5 kDa, 1 kDa, 2 kDa, 3 kDa, 4 kDa, 5 kDa, 6 kDa, 7.5 kDa, or 10 kDa. In some embodiments, the average molecular weight is less than about 100 kDa, 50 kDa, 25 kDa, 18 kDa, 17 kDa, 16 kDa, 15 kDa, 14 kDa, 13 kDa, 12 kDa, 11 kDa or 10 kDa. In some embodiments, the average molecular weight is about 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa or 15 kDa.

The partially desulfated heparins used in the pharmaceutical compositions described herein have reduced anti-coagulant activity as compared to unfractionated heparin. In typical embodiments, for example, the partially desulfated heparin has no more than 40% of the anti-coagulant activity of an equal weight of unfractionated heparin. These partially desulfated heparins are referred to herein as “low-anticoagulant heparins.” In certain embodiments, the low-anticoagulant heparin has no more than 35%, no more than 30%, no more than 25%, no more than 20%, even no more than 10%, 9%, 8%, 7%, 6%, 5%, 4%, 3%, 2%, or 1% of the anti-coagulant activity of an equal weight of unfractionated heparin.

Anticoagulant activity can be determined using assays known in the art. In certain embodiments, anticoagulant activity is determined by activated partial thromboplastin time (aPTT) assay. In some embodiments, anticoagulant activity is determined by assay of prothrombin time. In particular embodiments, anticoagulant activity is determined by anti-Xₐ activity. In a variety of embodiments, anticoagulant activity is determined by clotting assay. In some embodiments, anticoagulant activity is determined by amidolytic assays. In certain embodiments, anticoagulant activity is determined by the USP assay. See, e.g., U.S. Pat. No. 5,668,118, Example IV; Fryer et al., J. Pharmacol. Exp. Ther. 282: 208-219 (1997), at page 209; Rao et al., Am. J. Physiol. 299:C97-C110 (2010), at page C98; United States Pharmacopeia Convention 1995 (for USP anti-coagulant assay and amidolytic assay).

Partially desulfated heparins used in the pharmaceutical compositions described herein are low-anticoagulant in at least one of the above-described assays. In certain embodiments, the low-anticoagulant heparin used in the methods described herein is low-anticoagulant in more than one of the above-described assays. In a variety of embodiments, the low-anticoagulant heparin is one which exhibits substantially reduced anti-Xₐ activity, which can be determined in an assay carried out using plasma treated with Russell viper venom.

In specific embodiments, the partially desulfated heparin used in the pharmaceutical compositions described herein is the low-anticoagulant heparin derivative, ODSH. ODSH has been demonstrated to exhibit less than 9 U of anti-coagulant activity/mg in the USP anti-coagulant assay (e.g., 7±0.3 U), less than 5 U of anti-Xₐ activity/mg (e.g., 1.9±0.1 U/mg) and less than 2 U of anti-IIₐ activity/mg (e.g., 1.2±0.1 U/mg). Unfractionated heparin has an activity of 165-190 U/mg in all three assays. See Rao et al., Am. J. Physiol. 299:C97-C110 (2010), page C101. In addition, ODSH has a low affinity for anti-thrombin III (Kd˜339 μM or 4 mg/ml vs. 1.56 μM or 22 μg/ml for unfractionated heparin), consistent with the observed low level of anti-coagulant activity, measured as described in Rao et al., supra, at page C98.

ODSH can be prepared from any mammalian heparin, for example, bovine or porcine heparin. In an exemplary method of preparing ODSH from porcine heparin, ODSH is synthesized by cold alkaline hydrolysis of USP porcine intestinal heparin, which removes the 2-O and 3-O sulfates, leaving N- and 6-O sulfates on D-glucosamine sugars and carboxylates on α-L-iduronic acid sugars substantially intact. See Fryer, A. et al., J. Pharmacol. Exp. Ther. 282: 208-219 (1997). Using this method, ODSH can be produced with an average molecular weight of about 11.7±0.3 kDa, and low affinity for anti-thrombin III (Kd=339 μM or 4 mg/ml vs. 1.56 μM or 22 μg/ml for heparin), consistent with the observed low level of anticoagulant activity.

Methods for the preparation of 2-O, 3-O desulfated heparins may also be found, for example, in U.S. Pat. Nos. 5,668,118, 5,912,237, and 6,489,311, and WO 2009/015183, the contents of which are incorporated herein in their entirety, and in U.S. Pat. Nos. 5,296,471, 5,969,100, and 5,808,021. In one exemplary method, a partially desulfated heparin is made by cold alkaline hydrolysis. Heparin is obtained commercially at a grade, such as USP, sufficient for therapeutic application. The pH of the heparin solution is then raised to at least pH 13.0. The liquid solution may then be incubated for a period of time. Alternatively, in a preferred embodiment, the alkalinized solution is lyophilized and the dried lyophilate is incubated for a period of time. The lyophilate is then reconstituted with a suitable diluent for therapeutic application, or with a suitable solution for cation substitution.

4.1.2. Multivalent Cation

In the pharmaceutical compositions described herein, the partially desulfated heparins are associated with multivalent cations. The term “associated”, when used to describe the relationship between a partially desulfated heparin and a cation, means a chemically relevant association. The association may be as a salt, ion/counterion, complex, binding, coordination or any other chemically relevant association. The exact nature of the association will be readily apparent to a person of skill in the art depending on the form of the composition.

In various embodiments, the multivalent cations are selected from cations having a charge of +2, +3, +4, or greater. In some embodiments, the multivalent cation is an ion that contains both positive and negative charges, with a net charge greater than +1. Exemplary multivalent cations include metal ions, amino acids, and other organic and inorganic cations. In certain embodiments, the ion is a metal ion that is Zn²⁺, Ca²⁺, Mg²⁺ or Fe²⁺. In a specific embodiment, the cation is Ca²⁺. In another specific embodiment, the cation is Mg²⁺.

In certain embodiments, the partially desulfated heparins are associated primarily with one species of multivalent cation. In other embodiments, the partially desulfated heparins are associated with several different multivalent cation species. In specific embodiments, the partially desulfated heparins are associated with Mg²⁺ and Ca²⁺.

Multivalent cations may be introduced to the partially desulfated heparin composition at any step in the process for preparing partially or substantially desulfated heparins.

In one embodiment, the multivalent cation is present during alkaline hydrolysis of the heparin starting material. In certain embodiments, the multivalent cation is present as the chloride salt. In certain embodiments, the multivalent cation is present as the hydroxide salt. In one embodiment, the chloride salt is preferred for use during solution phase alkaline hydrolysis. In another embodiment, the hydroxide salt is preferred for use during solid phase alkaline hydrolysis. In another embodiment, the hydroxide salt is preferred for use when alkaline hydrolysis is performed as a paste.

Certain multivalent cations may affect the level of desulfation if present during alkaline hydrolysis, and may be used to achieve desired levels of desulfation. The amount of the multivalent cation may be titrated to control the amount of desulfation as described in U.S. Pat. No. 5,296,471 at Example 4.

Thus, when a multivalent cation is used during alkaline hydrolysis, the multivalent cation concentration used should be adjusted based on both the desired level of desulfation and the desired concentration of the final product. The molar multivalent cation concentration used during alkaline hydrolysis may be substantially less than the molar heparin concentration. Preferably, the molar ratio (multivalent cation:heparin) is about 0.1, 0.2, 0.3, 0.4, 0.5, 0.6, 0.7, 0.8, 0.9, 1.0, 1.1, 1.2, 1.3, 1.4, or 1.5, or any ranges composed of those values. Preferably, the concentration of the multivalent cation used during alkaline hydrolysis is about 0.01 mM, 0.05 mM, 0.1 mM, 0.5 mM, 1 mM, 5 mM, 10 mM, 20 mM, 50 mM, 100 mM, 250 mM, 500 mM or 1 M or any range composed of those numbers.

In certain embodiments, primarily monovalent cations are present during the cold alkaline hydrolysis step, and the multivalent cation is added later, during reconstitution of the lyophilate. In a most preferred embodiment, either MgCl₂ or CaCl₂ is added at high concentration during reconstitution of the lyophilate.

In certain embodiments, a method for preparing partially desulfated heparin comprises the steps of: (i) adjusting the pH of a solution of unfractionated heparin to at least 13; (ii) drying the alkaline solution of heparin of step (i); (iii) reconstituting the dried heparin with a solution comprising a multivalent cation; and, (iv) removing excess cations.

The multivalent cation concentration used during reconstitution may be equal to the concentration of the cation used during alkaline hydrolysis. Preferably, the multivalent cation concentration is at least about 2-fold, 3-fold, 4-fold, 5-fold, 6-fold, 7-fold, 8-fold, 9-fold, 10-fold, 15-fold, 20-fold, 25-fold, 30-fold, 50-fold, 75-fold, 100-fold, 150-fold, 200-fold, 250-fold, 500-fold, or 1000-fold the concentration of the cation used during alkaline hydrolysis. Preferably, the concentration of the multivalent cation used during reconstitution is about 0.1 M, 0.5 M, 1 M, 2 M, 3 M, 4 M, 5 M, or greater. Most preferably, the concentration is about 2 M.

Excess cations can be removed by any method known to those in the art. One preferred method of removing excess cations is the use of a desalting column. Another preferred method of removing excess cations is dialysis. After removal of excess ions, the solution preferably has about equal, 2-fold, 3-fold, 4-fold, 5-fold, 6-fold, 7-fold, 8-fold, 9-fold, 10-fold, 15-fold, 20-fold, 25-fold, 30-fold, 50-fold, 75-fold, 100-fold, 150-fold, 200-fold, 250-fold, 500-fold, or 1000-fold greater multivalent cation concentration to monovalent cation concentration. The solution may also be free or substantially free of monovalent cations.

4.1.3. Pharmaceutically Acceptable Carrier

The pharmaceutical composition further comprises a pharmaceutically acceptable carrier.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the subject. Some examples of materials which may serve as pharmaceutically acceptable carriers include: 1) sugars, such as lactose, glucose and sucrose; 2) starches, such as corn starch and potato starch; 3) cellulose, and its derivatives, such as sodium carboxymethyl cellulose, ethyl cellulose and cellulose acetate; 4) powdered tragacanth; 5) malt; 6) gelatin; 7) talc; 8) excipients, such as cocoa butter and suppository waxes; 9) oils, such as peanut oil, cottonseed oil, safflower oil, sesame oil, olive oil, corn oil and soybean oil; 10) glycols, such as propylene glycol; 11) polyols, such as glycerin, sorbitol, mannitol and polyethylene glycol; 12) esters, such as ethyl oleate and ethyl laurate; 13) agar; 14) buffering agents, such as Tris, HEPES, citrate, acetate, carbonate, tricine, and glycine-glycine; 15) alginic acid; 16) pyrogen-free water; 17) isotonic saline; 18) Ringer's solution; 19) ethyl alcohol; 20) phosphate buffer solutions; and 21) other non-toxic compatible substances employed in pharmaceutical formulations.

4.1.4. Further Characteristics

In typical embodiments, the final concentration of partially desulfated heparin in the pharmaceutical composition is between 0.1 mg/mL and 600 mg/mL. In certain embodiments, the final concentration of partially desulfated heparin in the pharmaceutical composition is between 200 mg/mL and 400 mg/mL.

In some embodiments, the concentration of partially desulfated heparin is greater than about 25 mg/mL. In certain embodiments, the concentration of partially desulfated heparin is greater than about 50 mg/mL. In a variety of embodiments, the concentration of partially desulfated heparin is greater than about 60 mg/mL, 70 mg/mL, 80 mg/mL, 90 mg/mL, or 100 mg/mL. In specific embodiments, the partially desulfated heparin is present in the pharmaceutical composition in a concentration greater than about 110 mg/mL, 120 mg/mL, 130 mg/mL, 140 mg/mL, 150 mg/mL, 160 mg/mL, 170 mg/mL, 180 mg/mL, or even greater than about 190 mg/mL or 200 mg/mL. In specific embodiments, the partially desulfated heparin is present in the pharmaceutical composition at a concentration of about 175 mg/mL. In another embodiment, the partially desulfated heparin is present in the pharmaceutical composition at a concentration of about 200 mg/mL. In one embodiment, the partially desulfated heparin is present in the pharmaceutical composition at a concentration of 400 mg/mL.

In certain embodiments, the concentration of partially desulfated heparin is 50 mg/mL to 500 mg/mL, 100 mg/mL to 400 mg/mL, or 150 mg/mL to 300 mg/mL. In specific embodiments, the concentration is 50 mg/mL, 100 mg/mL, 150 mg/mL, 200 mg/mL, 250 mg/mL, 300 mg/mL, 350 mg/mL, 400 mg/mL, 450 mg/mL or 500 mg/mL. In certain currently preferred embodiments, the concentration is 200 mg/mL, 300 mg/mL or 400 mg/mL.

In typical embodiments, the pharmaceutical composition has a viscosity of less than about 100 cP. In various embodiments, the pharmaceutical composition has a viscosity of less than about 80 cP. In certain embodiments, the pharmaceutical composition has a viscosity of less than about 60 cP. In particular embodiments, the pharmaceutical composition has a viscosity of less than about 20 cP.

In typical embodiments, the pharmaceutical composition has an osmolality less than about 2500 mOsm/kg. In various embodiments, the pharmaceutical composition has an osmolality between about 150 mOsm/kg and about 500 mOsm/kg. In certain embodiments, the pharmaceutical composition has an osmolality between about 275 mOsm/kg and about 300 mOsm/kg. In a particular embodiment, the pharmaceutical composition has an osmolality of about 285 mOsm/kg. In a specific embodiment, the pharmaceutical composition is isotonic.

In a variety of embodiments, the pharmaceutical composition further includes one or more pharmaceutically acceptable wetting agents, excipients, or diluents. The specific carriers, wetting agents, excipients, and/or diluents used will depend on the desired mode of administration.

In various embodiments, the pharmaceutical composition is in the form of a sterile, non-pyrogenic, fluid composition. In other embodiments, the pharmaceutical composition is in the form of a sterile, dry formulation. Pharmaceutical compositions of the present disclosure, suitable for administration to subjects, may optionally include additional active and/or therapeutic agents, as is known in the art. See Remington: The Science and Practice of Pharmacy, 21st Ed., Lippincott Williams & Wilkins (2005), incorporated herein by reference.

### 4.2. Unit Dosage Forms

The pharmaceutical compositions comprising partially desulfated heparins and multivalent cations described herein can be conveniently packaged in unit dosage forms suitable for desired routes of administration.

In various embodiments, the unit dosage forms contain 0.1 mg to 2 g, 0.2 mg to 800 mg, or 5 mg to 500 mg of partially desulfated heparin. In specific embodiments, the unit dosage form includes 10 mg, 50 mg, 100 mg, 200 mg, 500 mg, 800 mg, 1 g, 1.2 g, or 1.6 g of partially desulfated heparin.

In various embodiments, the unit dosage form comprises between 0.1 mL and 10 mL of the pharmaceutical composition. In a variety of embodiments, for example, the unit dosage form comprises 0.1 mL to 8 mL, 0.2 mL to about 5.0 mL, 0.5 mL to 5 mL, or 1 mL to 3 mL. Specific embodiments comprise 0.5 mL, 1 mL, 2 mL, 3 mL, 5 mL, 6 mL, or 8 mL.

In certain embodiments, the unit dosage form contains a pharmaceutical composition in which partially desulfated heparin is present in a concentration of between 0.1 mg/mL and 600 mg/mL. In certain embodiments, the partially desulfated heparin is present in a concentration of about 1 mg/ml to about 600 mg/mL.

In some embodiments, the concentration of partially desulfated heparin is greater than about 25 mg/mL. In certain embodiments, the concentration of partially desulfated heparin is greater than about 50 mg/mL. In a variety of embodiments, the concentration of partially desulfated heparin is greater than about 60 mg/mL, 70 mg/mL, 80 mg/mL, 90 mg/mL, or 100 mg/mL. In specific embodiments, the partially desulfated heparin is present in the pharmaceutical composition in a concentration greater than about 110 mg/mL, 120 mg/mL, 130 mg/mL, 140 mg/mL, 150 mg/mL, 160 mg/mL, 170 mg/mL, 180 mg/mL, or even greater than about 190 mg/mL or 200 mg/mL. In specific embodiments, the partially desulfated heparin is present in the pharmaceutical composition at a concentration of about 175 mg/mL. In another embodiment, the partially desulfated heparin is present in the pharmaceutical composition at a concentration of about 200 mg/mL. In one embodiment, the partially desulfated heparin is present in the pharmaceutical composition at a concentration of 400 mg/mL.

In certain embodiments, the concentration of partially desulfated heparin is 50 mg/mL to 500 mg/mL, 100 mg/mL to 400 mg/mL, or 150 mg/mL to 300 mg/mL. In specific embodiments, the concentration is 50 mg/mL, 100 mg/mL, 150 mg/mL, 200 mg/mL, 250 mg/mL, 300 mg/mL, 350 mg/mL, 400 mg/mL, 450 mg/mL or 500 mg/mL. In certain currently preferred embodiments, the concentration is 200 mg/mL, 300 mg/mL or 400 mg/mL.

Unit dosage form embodiments suitable for subcutaneous, intradermal, or intramuscular administration include preloaded syringes, autoinjectors, and autoinject pens, each containing a predetermined amount of the pharmaceutical composition described hereinabove.

In various embodiments, the unit dosage form is a preloaded syringe, comprising a syringe and a predetermined amount of the pharmaceutical composition of partially desulfated heparin described herein.

In certain preloaded syringe embodiments, the syringe is adapted for subcutaneous administration. In certain embodiments, the syringe is suitable for self-administration. In particular embodiments, the preloaded syringe is a single use syringe.

In various embodiments, the preloaded syringe contains about 0.1 mL to about 0.5 mL of the pharmaceutical composition. In certain embodiments, the syringe contains about 0.5 mL of the pharmaceutical composition. In specific embodiments, the syringe contains about 1.0 mL of the pharmaceutical composition. In particular embodiments, the syringe contains about 2.0 mL of the pharmaceutical composition.

In a variety of embodiments, the preloaded syringe comprises 0.2 mg to 800 mg of the partially desulfated heparin. In certain embodiments, the preloaded syringe comprises about 200 mg of the partially desulfated heparin.

In certain embodiments, the unit dosage form is an autoinject pen. The autoinject pen comprises an autoinject pen containing a pharmaceutical composition as described herein. In some embodiments, the autoinject pen delivers a predetermined volume of pharmaceutical composition. In other embodiments, the autoinject pen is configured to deliver a volume of pharmaceutical composition set by the user.

In various embodiments, the autoinject pen contains about 0.1 mL to about 5.0 mL of the pharmaceutical composition. In specific embodiments, the autoinject pen contains about 0.5 mL of the pharmaceutical composition. In particular embodiments, the autoinject pen contains about 1.0 mL of the pharmaceutical composition. In other embodiments, the autoinject pen contains about 5.0 mL of the pharmaceutical composition.

In a variety of embodiments, the concentration of partially desulfated heparin in the pharmaceutical composition contained within the autoinject pen is about 1 mg/ml to about 600 mg/mL. In some embodiments, the concentration of partially desulfated heparin is about 400 mg/mL. In specific embodiments, the concentration of partially desulfated heparin is about 200 mg/mL. In various embodiments, the concentration of partially desulfated heparin is about 175 mg/mL.

In various autoinject pen embodiments, the pen contains 0.2 mg to 800 mg of the partially desulfated heparin. In specific embodiments, the pen comprises about 200 mg of the partially desulfated heparin.

Unit dosage form embodiments include vials and ampules. In some embodiments of vials and ampules, the vial or ampule is suitable for delivering the pharmaceutical composition to a nebulizer or aerosolizer for administration by inhalation. In particular embodiments, the nebulizer is a jet nebulizer or an ultrasonic nebulizer.

### 4.3. Methods of Treatment

The pharmaceutical compositions described herein can be used for any of the therapeutic uses disclosed in U.S. Pat. Nos. 5,668,118, 5,707,974, 5,990,097, 6,489,311, 7,468,358; U.S. pre-grant publication nos. 2009/0054374, 2009/0036405, 2012/0196828, 2013/0303481; and international patent publication no. WO 01/19376, the disclosures of all of which are incorporated herein by reference in their entireties.

4.3.1. Attenuation of Myelosuppression

For example, in certain embodiments, the pharmaceutical compositions described herein can be used in a method of attenuating a myelosuppressive side effect of an antineoplastic treatment regimen, comprising adjunctively administering to a patient receiving an antineoplastic treatment regimen a pharmaceutical composition as described herein, in an amount effective to attenuate a myelosuppressive side effect of the antineoplastic treatment regimen. In various embodiments, the pharmaceutical composition is administered using one or more of the unit dosage forms described herein.

In specific embodiments, the partially desulfated heparin in the pharmaceutical composition is at least 90% desulfated at each of the 2-O and 3-O positions. In some embodiments, the partially desulfated heparin is at least 95% desulfated at each of the 2-O and 3-O positions.

In specific embodiments, the partially desulfated heparin is administered intravenously. In certain embodiments, the partially desulfated heparin is administered as one or more bolus injections. In certain embodiments, the partially desulfated heparin is administered as a continuous infusion. In certain embodiments, the partially desulfated heparin is administered as an intravenous bolus followed by a continuous intravenous infusion.

In specific embodiments, the partially desulfated heparin is administered subcutaneously.

In specific embodiments, the myelosuppressive side effect is thrombocytopenia. In some embodiments, the patient is diagnosed with grade 1, 2, 3, or 4 thrombocytopenia. In particular embodiments, the amount of partially desulfated heparin is sufficient to maintain platelet levels above levels that indicate grade 3 thrombocytopenia. In specific embodiments, the myelosuppressive side effect is neutropenia. In some embodiments, the patient is diagnosed with grade 1, 2, 3, or 4 neutropenia. The patient may have one or both of neutropenia and thrombocytopenia.

In specific embodiments, the antineoplastic treatment regimen comprises administering one or more chemotherapeutic agents. In certain embodiments, the one or more chemotherapeutic agents is selected from a folate antagonist, methotrexate and pemetrexed; a purine antagonist, cladribine, clofarabine, fludarabine, 6-mercaptopurine, nelarabine, pentostatin; a pyrimidine antagonist, capecitabine, cytarabine, 5-fluorouracil, gemcitabine, hydroxyurea; a biologic response modifier, interferon-alfa; bleomycin; a DNA alkylating agent, nitrosureas, carmustine, lomustine; a DNA cross-linking drug, a DNA alkylating agent, bendamustine, chlorambucil, cyclophosphamide, ifosfamide, mechlorethamine, melphalan, dacarbazine, temozolomide, procarbazine; asparaginase; an antibiotic, mitomycin; a platinum complex, carboplatin, cisplatin, oxaliplatin; a proteosome inhibitor, bortezomib; a spindle poison, a taxane, docetaxel, paclitaxel, nab-paclitaxel, a vinca, vinblastine, vincristine, vinorelbine; a topoisomerase inhibitor, an anthracycline, daunorubicin, daunomycin, doxorubicin, epirubicin, a camptothecine, irinotecan, topotecan, a podophyllotoxin, etoposide, teniposide, mitoxantrone; a tyrosine kinase inhibitor, erlotinib, gefitinib, imatinib, lapatinib, sorafenib, sunitinib; and ifosfamide. In specific embodiments, the antineoplastic treatment regimen comprises administration of gemcitabine and nab-paclitaxel.

In specific embodiments, the patient receives one or more therapeutic antibodies. In certain embodiments, the antibodies are selected from abciximab and rituximab.

In specific embodiments, the antineoplastic treatment regimen comprises administering two or more chemotherapeutic agents. In some embodiments, the two or more agents are selected from cisplatin and etoposide; carboplatin and etoposide; cisplatin and irinotecan; carboplatin and irinotecan; cyclophosphamide, doxorubicin (Adriamycin), and vincristine; cyclophosphamide/doxorubicin/vincristine; gemcitabine with vinorelbine or paclitaxel or nab-paclitaxel (Abraxane®); gemcitabine or capecitabine with oxaliplatin; cisplatin or carboplatin plus another chemotherapeutic agent; 5-fluorouracil with one or more of leucovorin, oxaliplatin, irinotecan. In certain embodiments, the two or more chemotherapeutic agents are administered according to a specific regimen is selected from 5FU Mayo, 5FU Roswell Park, LVFU2, FOLFOX4, FOLFOX6, bFOL, FUFOX, IFL, XELOX, XELIRI, CAPIRI, and ICE.

In specific embodiments, the patient receiving an antineoplastic treatment regimen has a cancer selected from the group consisting of: pancreatic cancer, ovarian cancer, uterine cancer, breast cancer, metastatic breast cancer, recurrent breast cancer, head and neck cancer, bladder cancer, urothelial cancer, lung cancer, colorectal cancer, gastric cancer, esophageal cancer, lymphoma, liver cancer, melanoma, prostate cancer, osteosarcoma, leukemia, acute myelogenous leukemia (AML) and pediatric acute lymphoblastic leukemia. In some embodiments, the cancer is a pediatric cancer.

In specific embodiments, the partially desulfated heparin has less anticoagulant activity than unfractionated heparin. In specific embodiments, the partially desulfated heparin has reduced affinity for anti-thrombin III as compared to unfractionated heparin. In specific embodiments, the partially desulfated heparin has reduced anti-Xa activity as compared to unfractionated heparin. In specific embodiments, the partially desulfated heparin is substantially non-anticoagulating. In specific embodiments, the partially desulfated heparin is produced by alkaline hydrolysis of unfractionated heparin.

In specific embodiments, the antineoplastic treatment regimen comprises radiation therapy. In certain embodiments, the radiation therapy is selected from x-ray radiation, gamma ray radiation, neutron radiation, proton radiation, external beam radiation therapy, and brachytherapy. In some embodiments, the radiation therapy further comprises one or more chemotherapeutic agents. In some embodiments, the radiation therapy is selected from x-ray radiation, gamma ray radiation, neutron radiation, proton radiation, external beam radiation therapy, and brachytherapy and the one or more chemotherapeutic agents is selected from a folate antagonist, methotrexate and pemetrexed; a purine antagonist, cladribine, clofarabine, fludarabine, 6-mercaptopurine, nelarabine, pentostatin; a pyrimidine antagonist, capecitabine, cytarabine, 5-fluorouracil, gemcitabine, hydroxyurea; a biologic response modifier, interferon-alfa; bleomycin; a DNA alkylating agent, nitrosureas, carmustine, lomustine; a DNA cross-linking drug, a DNA alkylating agent, bendamustine, chlorambucil, cyclophosphamide, ifosfamide, mechlorethamine, melphalan, dacarbazine, temozolomide, procarbazine; asparaginase; an antibiotic, mitomycin; a platinum complex, carboplatin, cisplatin, oxaliplatin; a proteosome inhibitor, bortezomib; a spindle poison, a taxane, docetaxel, paclitaxel, nab-paclitaxel, a vinca, vinblastine, vincristine, vinorelbine; a topoisomerase inhibitor, an anthracycline, daunorubicin, daunomycin, doxorubicin, epirubicin, a camptothecine, irinotecan, topotecan, a podophyllotoxin, etoposide, teniposide, mitoxantrone; a tyrosine kinase inhibitor, erlotinib, gefitinib, imatinib, lapatinib, sorafenib, and sunitinib.

In specific embodiments, the partially desulfated heparin is used for treating or preventing radiation-induced thrombocytopenia. In certain embodiments, the radiation is part of a treatment regimen. In certain embodiments, the radiation is not part of a treatment regimen.

In various embodiments, the patient is receiving a patient treatment regimen comprising one or more agent(s) selected from valproic acid, proton pump inhibitors, interferon, interferon-alpha, isotretinoin, panobinostat, thiazide diurectics, montelukast sodium (Singulair), quinidine, quinine, gold, sulfonamides, cephalothin, phenylbutazone, diphenylhydantoin, digitoxin and phenothiazine tranquilizers, heparin, and combinations thereof. In some embodiments, the patient treatment regiment comprises one or more agent(s) selected from cyclophosphamide, psychotropic drugs, anticonvulsants, clozapine, olanzapine, thionamides, ticlopidine, carbimazole, dapsone, dipyrone, methimazole, penicillin G, procainamide, propylthiouracil, trimethoprim, chloramphenicol, penicillins, cephalosporins, aminoglycosides, tetracyclines, nitroimidazoles, nitrofurantoin, flucytosine, rifampin, isoniazid, ethambutol, dapsone, sulfonamide antibiotics, clomiprimine, thiacetazone, dipyrone, sulfasalazine, mesalazine, ciprofloxacin, chloroquin, mebendazole, terbendafine, pyrimethamine, levamisole, ristocetin, griseofulvin, phenothiazines, benzodiazepines, amoxapine, meprobamate, barbiturates, risperidone, imipramine, desipramine, thiothixene, haloperidol, valproic acid, hydantoins, succinimides, trimethadione, carbamazepine, procainamide, quinidine, propafenone, captopril, propranolol, hydralazine, methyldopa, ibuprofen, indomethacin, sulindac, tolmetin, aspirin, aminopyine, phenylbutazone, diflunisal, benoxaprofen, allopurinol, colchicine, propylthiouracil, thiouracil, methimazole, carbimazole, thiocyanate, potassium perchlorate, cimetidine, ranatadine, tripelennamine, methaphenilene, thenalidine, mianserin, bromopheneramine, quinine, hydroxychloroquin, quinacrine, diazoxide, dihydropyridines, vesnarinone, aprindine, imipenem/cilastatin, zidovudine, fludarabine, acyclovir, turbinafine, aminoglutethimide, famotidine, bezafibrate, flutamide, tamoxafen, penicillamine, retinoic acid, metoclopramide, phenindone, dinitrophenol, ethacrynic acid, rauwolfia, ethanol, chlorpropamide, tolbutamide, thiazides, spironolactone, methazolamide, acetazolamide, levodopa and combinations thereof.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

4.3.2. Promotion of Thrombopoiesis

In certain embodiments, the pharmaceutical compositions described herein can be used in a method of promoting thrombopoiesis in a human subject, comprising administering an amount of a pharmaceutical composition as described herein effective to promote thrombopoiesis. In various embodiments, the pharmaceutical composition is administered using one or more of the unit dosage forms described herein.

In specific embodiments, the subject is thrombocytopenic. In some embodiments, the subject is diagnosed with thrombocytopenia selected from immune-mediated thrombocytopenia, drug-induced thrombocytopenia, or radiation-induced thrombocytopenia. In certain embodiments, the subject is diagnosed with immune thrombocytopenic purpura. In certain embodiments, the subject is diagnosed with radiation-induced thrombocytopenia. In certain embodiments the radiation-induced thrombocytopenia is caused by exposure to ionizing radiation, optionally non-therapeutic exposure to ionizing radiation.

In specific embodiments, the subject is non-thrombocytopenic.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

4.3.3. Promotion of Neutrophil Production

In certain embodiments, the pharmaceutical compositions described herein can be used in a method of promoting neutrophil production in a human subject, in which the method comprises administering an effective amount of a partially desulfated heparin to the subject.

In specific embodiments, the subject is neutropenic. In some embodiments, the subject is diagnosed with neutropenia selected from immune-mediated neutropenia, drug-induced neutropenia, or radiation-induced neutropenia. In certain embodiments, the subject is diagnosed with radiation-induced neutropenia. In certain embodiments, the radiation-induced neutropenia is caused by exposure to ionizing radiation, optionally non-therapeutic exposure to ionizing radiation.

In specific embodiments, the subject is non-neutropenic.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

4.3.4. Increasing Efficacy of Patient Treatment Regimens

In certain embodiments, the pharmaceutical compositions described herein can be used to increase efficacy of a patient treatment regimen having a myelosuppressive effect. The method comprises administering a therapeutically effective amount of a partially desulfated heparin to a patient as an adjunct to a patient treatment regimen having a myelosuppressive side effect, without reducing the dose and/or dosage frequency of the patient treatment regimen as compared to a reference treatment.

In specific embodiments, the administered dose of the patient treatment regimen is higher than the dose administered when the patient treatment regimen is administered in the absence of adjunctive administration of a partially desulfated heparin.

In specific embodiments, an amount of a partially desulfated heparin is administered sufficient to maintain platelet levels above levels that indicate grade 3 or grade 4 thrombocytopenia. Certain embodiments further comprise determining an initial platelet count in a blood sample from a human patient and administering an amount of a partially desulfated heparin effective to raise the patient's platelet count above a threshold level below which therapy with patient treatment regimen having a myelosuppressive side effect is contraindicated.

In specific embodiments, an amount of a partially desulfated heparin is administered sufficient to maintain neutrophil levels above levels that indicate grade 3 or grade 4 neutropenia. Certain embodiments further comprise: determining an initial neutrophil count in a blood sample from a human patient and administering an amount of a PF4-interacting heparinoid effective to raise the patient's neutrophil count above a threshold level below which therapy with patient treatment regimen having a myelosuppressive side effect is contraindicated.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

In specific embodiments, the patient is diagnosed with an autoimmune disease. In certain embodiments, the autoimmune disease is selected from rheumatoid arthritis, systemic lupus erythematosus, and multiple sclerosis.

In specific embodiments, the patient has a condition or disease causing impaired liver function. In certain embodiments, the patient is diagnosed with liver cancer, viral hepatitis, or liver cirrhosis.

4.3.5. Treatment and Prevention of Radiation Damage

In certain embodiments, the pharmaceutical compositions described herein can be used in a method of treating or preventing radiation damage in a subject exposed to whole-body radiation, comprising administering to a subject exposed to whole-body radiation a therapeutically or prophylactically effective amount of a pharmaceutical composition as described herein. In certain embodiments, the pharmaceutical compositions described herein can be used in a method of extending the life of a subject exposed to whole-body radiation, comprising administering to a subject exposed to whole-body radiation a therapeutically or prophylactically effective amount of a partially desulfated heparin. In various embodiments, the pharmaceutical composition is administered using one or more of the unit dosage forms described herein.

For example, in certain embodiments, the pharmaceutical compositions described herein can be used in a method of treating of acute radiation sickness (ARS). In some embodiments, the pharmaceutical compositions described herein can be used in a method of treating of radiation induced thrombocytopenia.

In specific embodiments, the partially desulfated heparin has an average molecular weight of about 8 kDa to about 15 kDa. In certain embodiments, the partially desulfated heparin has an average molecular weight of about 11 kDa to about 13 kDa.

In specific embodiments, the partially desulfated heparin is low-anti-coagulant.

In specific embodiments, the partially desulfated heparin in the pharmaceutical composition is at least 90% desulfated at each of the 2-O and 3-O positions. In some embodiments, the partially desulfated heparin is at least 95% desulfated at each of the 2-O and 3-O positions.

In specific embodiments, the partially desulfated heparin is administered parenterally. In specific embodiments, the partially desulfated heparin is administered intravenously or subcutaneously.

In specific embodiments, the subject is administered the partially desulfated heparin following exposure to whole-body radiation. In specific embodiments, the subject is administered the partially desulfated heparin within about 60 hours after exposure to whole-body radiation. In specific embodiments, the subject is administered the partially desulfated heparin about 2 hours or more after exposure to whole-body radiation.

In specific embodiments, the subject is administered the partially desulfated heparin prior to exposure to whole-body radiation.

In specific embodiments, the subject is exposed to whole-body radiation at a dose rate of about 0.1 Gy/min or greater. In specific embodiments, the subject is exposed to whole-body radiation at a dose rate of about 0.5 Gy/min or greater. In specific embodiments, the subject has a whole-body absorbed dose of radiation of about 2 Gy or greater. In specific embodiments, the subject has a whole-body absorbed dose of radiation of about 6 Gy or greater. In specific embodiments, the subject has a whole-body absorbed dose of radiation of about 8 Gy or greater.

In specific embodiments, the irradiation of the subject occurs over a time period of about 2 hours or less. In specific embodiments, the irradiation of the subject occurs over a time period of about 1 hour or less.

In specific embodiments, the partially desulfated heparin is administered in one or more doses. In specific embodiments, the one or more doses of the partially desulfated heparin are independently selected from about 1 mg/kg to about 40 mg/kg. In specific embodiments, the one or more doses of the partially desulfated heparin are independently selected from about 10 mg/kg to about 30 mg/kg.

In specific embodiments, the subject exposed to whole-body radiation has acute radiation syndrome. In specific embodiments, the subject exposed to whole-body radiation displays symptoms of hematopoietic, gastrointestinal and/or cerebrovascular syndromes. In specific embodiments, the symptoms include one or more of anemia, infection, bleeding, nausea, vomiting, diarrhea, severe dehydration, sepsis, and petechiae.

In specific embodiments, the methods further comprise administering one or more additional treatments. In specific embodiments, the one or more additional treatments are selected from a blood transfusion, antibiotics and a bone marrow transplant.

4.3.6. Treatment of Heparin-Induced Thrombocytopenia

In another exemplary use, the pharmaceutical compositions described herein are used to treat heparin-induced thrombocytopenia, as described in U.S. Pat. No. 7,468,358.

For example, in certain embodiments, the pharmaceutical compositions described herein can be used in a method of treating heparin-induced thrombocytopenia syndrome in a patient, comprising administering to said patient an effective amount of a partially desulfated heparin, without inducing platelet activation or thrombosis in the presence of heparin- and platelet factor 4-complex reactive antibodies.

As a further example, in certain embodiments, the pharmaceutical compositions described herein can be used in a method for the amelioration of platelet activation caused by heparin with HIT antibodies in a patient, comprising administering to said patient an effective amount of a partially desulfated heparin without inducing platelet activation or thrombosis in the presence of heparin—and platelet factor 4-complex reactive antibodies.

As a further example, in certain embodiments, the pharmaceutical compositions described herein can be used in a method for treating heparin-induced thrombocytopenia syndrome in a patient comprising administering to said patient an effective amount of a partially desulfated heparin without inducing platelet activation or thrombosis in the presence of heparin- and platelet factor 4-complex reactive and administering a drug selected from the group consisting of anti-thrombin drugs, anti-platelet drugs, and anti-inflammatory drugs.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

In specific embodiments, the partially desulfated heparin is administered by a method selected from the group consisting of intravenously, subcutaneously, inhalation, orally, and rectally.

4.3.7. Treatment of Asthma

In a further exemplary use, pharmaceutical compositions described herein are used to treat asthma. See U.S. Pat. No. 5,990,097, incorporated herein by reference in its entirety.

In typical embodiments, the composition is administered by inhalation. In certain embodiments, the composition is administered by intravenous administration. In some embodiments, the composition is administered by subcutaneous injection. In selected embodiments, acute treatment is administered by inhalation and/or intravenous administration, with chronic treatment continued thereafter via subcutaneous administration.

Pharmaceutical compositions comprising a partially desulfated heparin associated with Mg²⁺ as the multivalent cation are preferred for use in treating asthma or other forms of bronchoconstrictive or obstructive lung disease. Mg²⁺ is also a bronchodilator, and has been used for the treatment of asthma. See Coates et al., Respiratory Care, 56(3):314-318 (2011).

In various embodiments, further active agents are administered in combination with the multivalent cation-associated partially desulfated heparin.

In an exemplary embodiment, the pharmaceutical compositions are used for a method of treating a patient suffering from asthma comprising administering to the patient a pharmaceutical composition having an asthmatic response-reducing amount of a partially desulfated heparin.

A further exemplary embodiment provides for a method of preventing asthmatic response in a patient comprising administering to the patient, prior to any asthmatic response in the patient, a pharmaceutical composition having a pharmaceutically effective amount of a partially desulfated heparin.

In certain embodiments, the asthmatic response-reducing amount is from about 1 mg to about 100 mg per kilogram of the body weight of the patient.

In certain embodiments, the partially desulfated heparin has an average molecular weight of from about 100 to about 8000 Da. In various embodiments, the average molecular weight will be about 8-15 kDa. In certain embodiments, the average molecular weight will be about 11-13 kDa. In some embodiments, the average molecular weight is greater than 0.5 kDa, 1 kDa, 2 kDa, 3 kDa, 4 kDa, 5 kDa, 6 kDa, 7.5 kDa, or 10 kDa. In some embodiments, the average molecular weight is less than about 100 kDa, 50 kDa, 25 kDa, 18 kDa, 17 kDa, 16 kDa, 15 kDa, 14 kDa, 13 kDa, 12 kDa, 11 kDa or 10 kDa. In some embodiments, the average molecular weight is about 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa or 15 kDa. In some embodiments, the minimum molecular weight is greater than about 0.5 kDa, 1 kDa, 2 kDa, 3 kDa, 4 kDa, 5 kDa, 6 kDa, 7.5 kDa, or 10 kDa. In various embodiments, the minimum molecular weight is about 2 kDa.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

In specific embodiments, the patient is suffering from antigen-induced asthma.

In specific embodiments, the method reduces asthmatic response at least by reducing airways hyperactivity in the patient. In specific embodiments, the method reduces asthmatic response at least by increasing the activity of desensitized M2 muscarinic receptors in the patient. In specific embodiments, the method reduces asthmatic response at least by reducing bronchoconstriction in the patient. In specific embodiments, the method reduces asthmatic response at least by reducing airway smooth muscle cell proliferation in the patient. In specific embodiments, the method reduces asthmatic response at least by inhibiting complement-mediated hemolysis in the patient.

In specific embodiments, the patient has had a history of asthmatic reaction.

In specific embodiments, the method prevents asthmatic response at least by reducing airways hyperactivity in the patient. In specific embodiments, the method prevents asthmatic response at least by increasing the activity of desensitized M2 muscarinic receptors in the patient. In specific embodiments, the method prevents asthmatic response at least by reducing bronchoconstriction in the patient. In specific embodiments, the method prevents asthmatic response at least by reducing airway smooth muscle cell proliferation in the patient. In specific embodiments, the method prevents asthmatic response at least by inhibiting complement-mediated hemolysis in the patient.

4.3.8. Treatment of Lung Disorders

In a further exemplary use, other lung disorders are treated using the pharmaceutical compositions described herein. Exemplary lung disorders include chronic bronchitis, emphysema, acute respiratory distress syndrome, cystic fibrosis, bacterial infections and viral infections.

For example, in certain embodiments, the pharmaceutical compositions described herein are used to treat a bacterial lung infection, comprising: administering a therapeutically effective amount of a partially desulfated heparin to a subject suffering from a bacterial lung infection.

In specific embodiments, the infecting bacteria are Gram positive bacteria. In other embodiments, the infecting bacteria are Gram negative bacteria. In particular embodiments, the bacterial lung infection is a Pseudomonas infection. In some embodiments, the Pseudomonas infection is chronic.

In specific embodiments, the subject is suffering from cystic fibrosis.

In specific embodiments, the partially desulfated heparin is administered parenterally. In particular embodiments, the partially desulfated heparin administered subcutaneously, intravenously, or by inhalation.

In specific embodiments, the partially desulfated heparin is administered adjunctively to a second therapeutic agent. In particular embodiments, the second therapeutic agent is selected from the group consisting of: (a) an anti-microbial agent, (b) a DNase, (c) a bronchodilator, (d) a mucolytic agent, and combinations thereof. In some embodiments, the partially desulfated heparin and the second therapeutic agent are administered via the same route. In some embodiments, the partially desulfated heparin and the second therapeutic agent are administered concurrently. In some embodiments, the partially desulfated heparin and the second therapeutic agent are administered simultaneously. In some embodiments, the partially desulfated heparin and the second therapeutic agent are administered sequentially. In some embodiments, the partially desulfated heparin and the second therapeutic agent are administered separately.

In specific embodiments, the partially desulfated heparin and the second therapeutic agent are formulated in a single dosage form. In specific embodiments, the partially desulfated heparin and the second therapeutic agent are formulated in separate dosage forms.

In specific embodiments, the partially desulfated heparin and the second therapeutic agent are administered via different routes.

In certain embodiments the subject is suffering from chronic obstructive pulmonary disease. In certain embodiments, a second therapeutic agent is selected from the group consisting of: (a) an anti-microbial agent, (b) a bronchodilator, (d) a mucolytic agent, (e) an anti-inflammatory agent, and combinations thereof.

In specific embodiments, the Pseudomonas infection is acute.

In specific embodiments, the subject is hospitalized. In some embodiments, the subject is intubated. In some embodiments, the subject is on a ventilator.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

4.3.9. Improvement of Lung Function in Cystic Fibrosis

In certain embodiments, the pharmaceutical compositions described herein can be used in a method of improving lung function in a subject suffering from cystic fibrosis, comprising: administering to said subject a therapeutically effective amount of a partially desulfated heparin and a DNase.

In a certain embodiments, the DNase is a recombinant human deoxyribonuclease I. In a specific embodiment, the recombinant human deoxyribonuclease I is dornase alfa.

In some embodiments, an anti-microbial agent is used with the DNase and partially desulfated heparin. In specific embodiments, the anti-microbial agent is selected from tobramycin, aztreonam, ciprofloxacin, a fluoroquinolone, and levofloxacin. In certain embodiments, a bronchodilator is used with the DNase and partially desulfated heparin. In certain embodiments, a mucolytic agent is used with the DNase and partially desulfated heparin.

In certain embodiments, the partially desulfated heparin and the DNase are formulated for inhalation using the same device.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

4.3.10. Treatment and Prevention of Rhinitis

In a further exemplary use, rhinitis is treated using the pharmaceutical compositions described herein. In specific embodiments, the pharmaceutical compositions described herein are used in a method of treating or preventing rhinitis in a subject, comprising administering to a subject a therapeutically or prophylactically effective amount of a pharmaceutical composition as described herein.

In some embodiments, the rhinitis is allergic. In some embodiments, the rhinitis is non-allergic. Is certain embodiments, the rhinitis is hormonal, medication-induced or irritant-induced. In specific embodiments, the rhinitis is vasomomotor rhinitis, infectious rhinitis, rhinitis medicamentosa, chronic atrophic rhinitis, rhinitis sicca, polypous rhinitis or hypertrophic rhinitis. In some embodiments, the rhinitis is inflammatory. In some embodiments, rhinitis is non-inflammatory. In specific embodiments, the infectious rhinitis is viral. In specific embodiments, the infectious rhinitis is bacterial. In specific embodiments, the rhinitis is hay fever.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

In certain embodiments, the partially desulfated heparin is administered adjunctively to a second therapeutic agent. In some embodiments, the second therapeutic agent is an antihistamine, corticosteroid, decongestant, leukotriene inhibitor or antibiotic. In specific embodiments, the antihistamine is selected from brompheniramine, chlorpheniramine, carbinoxamine, clemastine, dimenhydrinate, diphenhydramine, promethazine, hydroxyzine, cyproheptadine, acrivastine, azelastine, olopatadine, cetirizine, levocetirizine, desloratadine, fexofenadine, loratadine and emedastine. In specific embodiments, the decongestant is selected from phenylephrine, pseudoephedrine, oxymetazoline, levmetamfetamine and propylhexedrine. In specific embodiments, the corticosteroid is selected from fluticasone, beclomethasone, budesonide, ciclesonide, flunisolide, mometasone, and triamcinolone. In specific embodiments, the leukotriene inhibitor is selected from montelukast, zafirlukast and zileuton.

In certain embodiments, the multivalent cation is therapeutically or prophylactically effective. In specific embodiments, the multivalent cation is Mg²⁺.

In certain embodiments, the partially desulfated heparin is administered by inhalation. In certain embodiments, the partially desulfated heparin is administered intranasally.

4.3.11. Treatment and Prevention of Sinusitis

In a further exemplary use, sinusitis is treated using the pharmaceutical compositions described herein. In specific embodiments, the pharmaceutical compositions described herein are used in a method of treating or preventing sinusitis in a subject, comprising administering to a subject a therapeutically or prophylactically effective amount of a pharmaceutical composition as described herein.

In certain embodiments, the sinusitis is caused by an allergy, infection or an autoimmune disorder. In specific embodiments, the infection is viral. In certain embodiments, the viral sinusitis is caused by influenza virus, coronavirus, rhinovirus or respiratory syncytial virus. In specific embodiments, the infection is bacterial. In certain embodiments, the bacterial sinusitis is caused by streptococcus pneumoniae or haemophilus influenza. In certain embodiments, the sinusitis is acute, subacute, chronic or recurrent sinusitis. In specific embodiments, the sinusitis is rhinosinusitis.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

In certain embodiments, the partially desulfated heparin is administered adjunctively to a second therapeutic agent. In some embodiments, the second therapeutic agent is an antihistamine, corticosteroid, decongestant, leukotriene inhibitor or antibiotic. In certain embodiments, the partially desulfated heparin is administered adjunctive to surgery. In certain embodiments, the partially desulfated heparin is administered adjunctive to nasal wash.

In specific embodiments, the antihistamine is selected from brompheniramine, chlorpheniramine, carbinoxamine, clemastine, dimenhydrinate, diphenhydramine, promethazine, hydroxyzine, cyproheptadine, acrivastine, azelastine, olopatadine, cetirizine, levocetirizine, desloratadine, fexofenadine, loratadine and emedastine. In specific embodiments, the decongestant is selected from phenylephrine, pseudoephedrine, oxymetazoline, levmetamfetamine and propylhexedrine. In specific embodiments, the corticosteroid is selected from fluticasone, beclomethasone, budesonide, ciclesonide, flunisolide, mometasone, triamcinolone. In specific embodiments, the leukotriene inhibitor is selected from montelukast, zafirlukast and zileuton.

In certain embodiments, the multivalent cation is therapeutically or prophylactically effective. In specific embodiments, the multivalent cation is Mg²⁺.

In certain embodiments, the partially desulfated heparin is administered by inhalation. In certain embodiments, the partially desulfated heparin is administered intranasally.

4.3.12. Treatment of Inflammatory Disorders

In another aspect, the pharmaceutical compositions described herein are useful for the treatment of inflammation or inflammatory disorders. See, e.g., Rao et al., Am. J. Physiol. Cell Physiol. 299:C97-C110 (2010). Exemplary, non-limiting, inflammatory disorders include asthma, rheumatoid arthritis, multiple sclerosis, Crohn's disease, ulcerative colitis, and inflammatory bowel disease.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

4.3.13. Treatment of Ischemia Reperfusion Injury

In a further exemplary use, the pharmaceutical compositions described herein are useful for treating ischemia-reperfusion injury. See U.S. Pat. No. 6,489,311, incorporated herein by reference in its entirety.

For example, in certain embodiments, the pharmaceutical compositions described herein are used in a method for inhibiting apoptosis in ischemic-reperfused myocardium comprising administering to a human in need thereof from 3 mg/kg to 100 mg/kg of a partially desulfated heparin to reduce apoptosis cell death in myocardial infarction.

In specific embodiments, the administering inhibits apoptosis in ischemic-reperfused brain, reducing neuronal cell death in stroke. In specific embodiments, the administering inhibits apoptosis in the failing heart, reducing apoptosis cell death in congestive heart failure and cardiomyopathy.

In further exemplary embodiments, the pharmaceutical compositions described herein are used in a method for inhibiting apoptosis in ischemic-reperfused cyocardium comprising administering to a human in need thereof an effective amount of partially desulfated heparin to reduce apoptosis cell death in myocardial infarction.

In further exemplary embodiments, the pharmaceutical compositions described herein are used in a method for enhancing the anti-apoptotic effect of a partially desulfated heparin comprising conjugating the partially desulfated heparin to a lipophilic moiety by reaction across a carboxylic acid or free amine group to enhance cellular uptake by cell types not normally concentrating heparin.

In specific embodiments, the partially desulfated heparin is an O-desulfated heparin. In certain embodiments, the partially desulfated heparin is a N-desulfated heparin.

In specific embodiments, the partially desulfated heparin has an average molecular weight of about 8 kDa to about 15 kDa, of about 11 kDa to about 14 kDa, or of about 11 kDa to about 13 kDa.

In specific embodiments, the partially desulfated heparin is low-anti-coagulant.

In specific embodiments, the partially desulfated heparin is substantially desulfated at the 2-O position or the 3-O position. In particular embodiments, the partially desulfated heparin is substantially 2-O, 3-O desulfated.

### 4.4. Modes of Administration

The pharmaceutical compositions described herein can be formulated for administration to subjects by a variety of routes, including intranasally, by inhalation, intramuscularly, intraperitoneally, and parenterally, including intravenously or subcutaneously. The pharmaceutical compositions can be formulated in volumes and concentrations suitable for bolus administration, for continuous infusion, or for subcutaneous administration.

The multivalent cation compositions of the present disclosure permit higher concentrations of a partially desulfated heparin to be administered to the subject. Higher concentrations are useful for all forms of administration, particularly those where the heparin is administered over a short time. Exemplary short administrations are bolus intravenous administration, subcutaneous, intramuscular administration and inhalation. In one preferred embodiment, the pharmaceutical compositions are suitable for subcutaneous administration. In another preferred embodiment, the pharmaceutical compositions are suitable for inhalation.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

Partially desulfated heparins can be administered in the methods described herein by a variety of routes, as noted above. In particular embodiments, the partially desulfated heparin is administered subcutaneously and/or by inhalation.

### 4.5. Schedule of Administration

The schedule of administration will vary depending upon the route of administration and the desired result. Partially desulfated heparins may be administered to the subject in an amount sufficient or effective to provide a therapeutic benefit, i.e., a therapeutically effective amount, and/or a preventative benefit, i.e., prophylactically effective amount. The therapeutically effective amount and prophylactically effective amount depend in part on the severity of the disorder, the extent of damage or injury and other characteristics of the subject to be treated, e.g., age, size, etc.

One or more doses of partially desulfated heparin may be independently selected from about 1 mg/kg to about 40 mg/kg. In particular, one or more doses may be independently selected from about 10 mg/kg to about 30 mg/kg. Doses may be selected from 1 mg/kg, 2 mg/kg, 8 mg/kg and 25 mg/kg.

The one or more doses of partially desulfated heparin may be independently selected from partially desulfated heparins that differ in structure and composition. For example, in certain embodiments, the subject receives one or more doses of ODSH as well as one or more doses of a different low anticoagulant heparin. In certain embodiments, the patient is also administered one or more doses of unfractionated, or other anticoagulating, heparins. The mixture of low anticoagulant heparin with unfractionated heparin permits some anti-coagulation activity to be administered simultaneous to the treatment using a low anticoagulant heparin.

In certain embodiments, partially desulfated heparin is administered to a subject following the subject's exposure to a discrete insult. The insult does not need to be instantaneous, but should occur over a short period of time. The subject may be administered one or more doses of partially desulfated heparin within about 60 hours following the discrete insult. For example, the partially desulfated heparin may be administered at about 4 hours, about 16 hours, or about 28 hours after the discrete insult. For example, the partially desulfated heparin may be administered at about 24 hours, about 36 hours or about 48 hours after the discrete insult. The subject may also be administered a dose immediately, or as soon as possible, after the beginning of the insult. Administration may occur before, during or after the discrete insult.

In certain embodiments, partially desulfated heparin is administered to a subject during an ongoing insult the subject. Ongoing insults may or may not have a defined beginning or end. An ongoing insult occurs over a longer period of time. The subject may be administered one or more doses of partially desulfated heparin at any interval during the insult, including every 4 hours, 8 hours, 12 hours, 24 hours, or 48 hours. Administration may occur before, during or after the ongoing insult.

Dosing of partially desulfated heparin may continue for multiple days or weeks such as 2 days or more, 3 days or more, 4 days or more, 5 days or more, 6 days or more, 1 week or more, 2 weeks or more, 3 weeks or more or 4 weeks or more. Dosing may also be of indefinite duration.

The partially desulfated heparin may be administered continuously over a period of time such as from about 1 minute to 10 hours. For example, the subject may receive an intravenous infusion of partially desulfated heparin continuously over a period of about 2 hours or more, about 3 hours or more, about 4 hours or more, about 5 hours or more, about 6 hours or more, about 7 hours or more, or about 8 hours or more. Preferably, the subject may use a nebulizer administering the partially desulfated heparin continuously over a period of about 1 minute, 2 minutes, 3 minutes, 4 minutes, 5 minutes, 6 minutes, 7 minutes, 8 minutes, 9 minutes, 10 minutes, 11 minutes or more. The continuous administration of partially desulfated heparin may occur before, after and/or during a discrete or ongoing insult. There may be multiple sessions of continuous administration, such as 3 or more sessions of continuous administration. For example, a subject may receive a session of continuous administration for 5 minutes or more for each 24 hour period following the start of a treatment regimen. The partially desulfated heparin may be administered over very short periods, such as by subcutaneous injection. For these periods, the length of time of administration is negligible relative to the dosing interval.

### 4.6. Additional Descriptions of Embodiments

In addition to the above, the following embodiments are described.

(1) A pharmaceutical composition comprising a partially desulfated heparin, a multivalent cation, and a pharmaceutically acceptable carrier.

(2) The pharmaceutical composition of embodiment (1), wherein the partially desulfated heparin is at least about 50% desulfated at the 2-O position.

(3) The pharmaceutical composition of embodiment (2), wherein the partially desulfated heparin is at least about 99% desulfated at the 2-O position.

(4) The pharmaceutical composition of embodiment (1), wherein the partially desulfated heparin is at least about 50% desulfated at the 3-O position.

(5) The pharmaceutical composition of embodiment (4), wherein the partially desulfated heparin is at least about 99% desulfated at the 3-O position.

(6) The pharmaceutical composition of embodiment (1), wherein the partially desulfated heparin is at least about 50% desulfated at each of the 2-O and 3-O positions.

(7) The pharmaceutical composition of embodiment (6), wherein the partially desulfated heparin is at least about 99% desulfated at each of the 2-O and 3-O positions.

(8) The pharmaceutical composition of embodiment (1), wherein the partially desulfated heparin is ODSH.

(9) The pharmaceutical composition of any of embodiments (1) to (8), wherein the partially desulfated heparin comprises substantially N-sulfated and 6-O sulfated D-glucosamine and substantially intact carboxylates of α-L-iduronic acid.

(10) The pharmaceutical composition of any of embodiments (1) to (9) wherein the partially desulfated heparin has an average molecular weight of greater than about 2 kDa.

(11) The pharmaceutical composition of embodiment (10), wherein the partially desulfated heparin has an average molecular weight of about 8 kDa to about 15 kDa.

(12) The pharmaceutical composition of embodiment (11), wherein the partially desulfated heparin has an average molecular weight of about 11 kDa to about 13 kDa.

(13) The pharmaceutical composition of any of embodiments (1) to (12), wherein the multivalent cation is a divalent cation.

(14) The pharmaceutical composition of embodiment (13), wherein the divalent cation is Mg²⁺.

(15) The pharmaceutical composition of embodiment (13), wherein the divalent cation is Ca²⁺.

(16) The pharmaceutical composition of any of embodiments (1) to (15), wherein the multivalent cation is at a concentration of at least about equal to the concentration of a monovalent cation.

(17) The pharmaceutical composition of embodiment (16), wherein the multivalent cation is at a concentration of at least about twice the concentration of the monovalent cation.

(18) The pharmaceutical composition of embodiment (17), wherein the multivalent cation is at a concentration of at least about ten times the concentration of the monovalent cation.

(19) The pharmaceutical composition of embodiment (18), wherein the pharmaceutical composition is substantially free of the monovalent cation.

(20) The pharmaceutical composition of any of embodiments (16) to (19), wherein the monovalent cation is Na⁺, Li⁺, K⁺, or combinations thereof.

(21) The pharmaceutical composition of embodiment (20), wherein the monovalent cation is Na⁺.

(22) The pharmaceutical composition of any of embodiments (1) to (21), wherein the partially desulfated heparin is at a concentration of between about 1 mg/mL and about 600 mg/mL.

(23) The pharmaceutical composition of embodiment (22), wherein the partially desulfated heparin is at a concentration of at least about 50 mg/mL.

(24) The pharmaceutical composition of embodiment (22), wherein the partially desulfated heparin is at a concentration of about 175 mg/mL.

(25) The pharmaceutical composition of embodiment (22), wherein the partially desulfated heparin is at a concentration of about 200 mg/mL.

(26) The pharmaceutical composition of embodiment (22), wherein the partially desulfated heparin is at a concentration of about 400 mg/mL.

(27) The pharmaceutical composition of any of embodiments (1) to (21), wherein the pharmaceutical composition has an osmolality less than about 2500 mOsm/kg.

(28) The pharmaceutical composition of embodiment (27), wherein the pharmaceutical composition has an osmolality between about 150 mOsm/kg and about 500 mOsm/kg.

(29) The pharmaceutical composition of embodiment (28), wherein the pharmaceutical composition has an osmolality between about 275 mOsm/kg and about 300 mOsm/kg.

(30) The pharmaceutical composition of embodiment (29), wherein the pharmaceutical composition has an osmolality of about 285 mOsm/kg.

(31) The pharmaceutical composition of embodiment (27), wherein the pharmaceutical composition is isotonic.

(32) The pharmaceutical composition of any of embodiments (1) to (21), wherein the pharmaceutical composition has a viscosity of less than about 100 cP.

(33) The pharmaceutical composition of embodiment (32), wherein the pharmaceutical composition has a viscosity of less than about 80 cP.

(34) The pharmaceutical composition of embodiment (33), wherein the pharmaceutical composition has a viscosity of less than about 60 cP.

(35) The pharmaceutical composition of embodiment (34), wherein the pharmaceutical composition has a viscosity of less than about 20 cP.

(36) The pharmaceutical composition of any of embodiments (1) to (35), wherein the partially desulfated heparin is of mammalian origin.

(37) The pharmaceutical composition of embodiment (36), wherein the partially desulfated heparin is of bovine origin.

(38) The pharmaceutical composition of embodiment (36), wherein the partially desulfated heparin is of porcine origin.

(39) The pharmaceutical composition of any of embodiments (1) to (38), wherein the pharmaceutical composition further comprises a buffering agent.

(40) The pharmaceutical composition of embodiment (39), wherein the buffering agent is selected from the group consisting of: Tris, HEPES, citrate, phosphate, acetate, carbonate, tricine, and glycine-glycine.

(41) The pharmaceutical composition of embodiment (40), wherein the buffering agent is Tris.

(42) The pharmaceutical composition of any of embodiments (1) to (41), wherein the pharmaceutical composition further comprises one or more of a wetting agent, a stabilizing agent, and a preservative.

(43) The pharmaceutical composition of any of embodiments (1) to (42), wherein the composition is a liquid formulation.

(44) The pharmaceutical composition of any of embodiments (1) to (42), wherein the composition is a dry formulation.

(45) The pharmaceutical composition of embodiment (44), wherein the dry formulation is a lyophilate.

(46) The pharmaceutical composition of embodiment (43), wherein the liquid formulation is in a form suitable for subcutaneous injection.

(47) The pharmaceutical composition of embodiment (43), wherein the liquid formulation is in a form suitable for nebulization.

(48) The pharmaceutical composition of embodiment (43) or (44), wherein the composition in a form suitable for inhalation.

(49) The pharmaceutical composition of any of embodiments (1) to (48), wherein the composition is stable at ambient temperature for at least 1 year.

(50) The pharmaceutical composition of embodiment (49), wherein the composition is stable at ambient temperature for at least 2 years.

(51) The pharmaceutical composition of embodiment (50), wherein the composition is stable at ambient temperature for at least 5 years.

(52) A method for preparing a partially desulfated heparin associated with a multivalent cation, comprising the steps of:
(i) adjusting the pH of a solution of unfractionated heparin to at least 13;
(ii) drying the alkaline solution of heparin of step (i);
(iii) reconstituting the dried heparin with a solution comprising a multivalent cation; and,
(iv) removing excess cations.

(53) The method of embodiment (52), wherein excess cations are removed by contacting the solution with a desalting resin.

(54) The method of embodiment (52), wherein excess cations are removed by dialyzing the solution.

(55) The method of embodiment (54), wherein the molecular weight cutoff for dialysis is about 2000 Da.

(56) The method of any of embodiments (52) to (55), wherein the multivalent cation of step (iii) is a divalent cation.

(57) The method of embodiment (56), wherein the divalent cation is Mg²⁺.

(58) The method of embodiment (56), wherein the divalent cation is Ca²⁺.

(59) The method of any of embodiments (52) to (58), further comprising at least one monovalent cation in the solution of step (i).

(60) The method of any of embodiments (52) to (58), further comprising at least one multivalent cation in the solution of step (i).

(61) The method of any of embodiments (52) to (58), further comprising at least one monovalent cation and at least one multivalent cation in the solution of step (i).

(62) The method of embodiment (60) or (61), wherein the multivalent cation of step (i) is Ca²⁺ or Mg²⁺.

(63) The method of any of embodiments (52) to (62), wherein the solution of step (i) comprises a reducing agent.

(64) The method of embodiment (63), wherein the reducing agent is sodium borohydride.

(65) The method of any of embodiments (52) to (64), wherein the multivalent cation in step (iii) is at a concentration of at least about 0.5M.

(66) The method of embodiment (65), wherein the multivalent cation in step (iii) is at a concentration of at least about 1M.

(67) The method of embodiment (66), wherein the multivalent cation in step (iii) is at a concentration of at least about 2M.

(68) The method of any of embodiments (52) to (67), wherein the multivalent cation in step (iii) is at a concentration of at least twice the concentration of a monovalent cation.

(69) The method of embodiment (68), wherein the multivalent cation in step (iii) is at a concentration of at least ten times the concentration of the monovalent cation.

(70) The method of any of embodiments (52) to (69), wherein the concentration of multivalent cation in step (iii) is in sufficient excess to replace monovalent cations associated with the partially desulfated heparin.

(71) The method of any of embodiments (52) to (70), further comprising the step of concentrating the partially desulfated heparin of step (iv).

(72) The method of embodiment (71), wherein the step of concentrating is performed by lyophilization and reconstitution.

(73) The method of any of embodiments (52) to (72), wherein the unfractionated heparin is of mammalian origin.

(74) The method of embodiment (73), wherein the unfractionated heparin is of bovine origin.

(75) The method of embodiment (73), wherein the unfractionated heparin is of porcine origin.

(76) The method of any of embodiments (52) to (75), wherein the partially desulfated heparin in the preparation is at a concentration of about 1 mg/mL to about 600 mg/mL.

(77) The method of embodiment (76), wherein the partially desulfated heparin in the preparation is at a concentration of at least about 50 mg/mL.

(78) The method of embodiment (77), wherein the partially desulfated heparin in the preparation is at a concentration of at least about 175 mg/mL.

(79) The method of embodiment (78), wherein the partially desulfated heparin in the preparation is at a concentration of at least about 200 mg/mL.

(80) The method of embodiment (79), wherein the partially desulfated heparin in the preparation is at a concentration of at least about 400 mg/mL.

(81) A product generated by the method of any of embodiments (52) to (80).

(82) The product of embodiment (81), wherein the partially desulfated heparin in the preparation is at least about 50% desulfated at the 2-O position.

(83) The product of embodiment (82), wherein the partially desulfated heparin in the preparation is at least about 99% desulfated at the 2-O position.

(84) The product of embodiment (81), wherein the partially desulfated heparin in the preparation is at least about 50% desulfated at the 3-O position.

(85) The product of embodiment (84), wherein the partially desulfated heparin in the preparation is at least about 99% desulfated at the 3-O position.

(86) The product of embodiment (81), wherein the partially desulfated heparin in the preparation is at least about 50% desulfated at each of the 2-O and 3-O positions.

(87) The product of embodiment (86), wherein the partially desulfated heparin in the preparation is at least about 99% desulfated at each of the 2-O and 3-O positions.

(88) The product of embodiment (81), wherein the partially desulfated heparin in the preparation is ODSH.

(89) The product of any of embodiments (81) to (88), wherein the partially desulfated heparin in the preparation comprises substantially N-sulfated and 6-O sulfated D-glucosamine and substantially intact carboxylates of α-L-iduronic acid.

(90) The product of embodiments (81) to (89), wherein the partially desulfated heparin in the preparation has an average molecular weight of greater than about 2 kDa.

(91) The product of embodiment (90), wherein the partially desulfated heparin in the preparation has an average molecular weight of about 8 kDa to about 15 kDa.

(92) The product of embodiment (91), wherein the partially desulfated heparin in the preparation has an average molecular weight of about 11 kDa to about 13 kDa.

(93) The product of any of embodiments (81) to (92), wherein the partially desulfated heparin in the preparation is associated with a divalent cation.

(94) The product of embodiment (93), wherein the divalent cation is Mg²⁺.

(95) The product of embodiment (93), wherein the divalent cation is Ca²⁺.

(96) The product of any of embodiments (81) to (95), wherein the multivalent cation is at an amount of at least about equal to the amount of a monovalent cation.

(97) The product of embodiment (96), wherein the multivalent cation is at an amount of at least about twice the amount of the monovalent cation.

(98) The product of embodiment (97), wherein the multivalent cation is at an amount of at least about ten times the amount of the monovalent cation.

(99) The product of embodiment (98), wherein the product is substantially free of the monovalent cation.

(100) The product of any of embodiments (96) to (99), wherein the monovalent cation is Na⁺, Li⁺, K⁺, or combinations thereof.

(101) The product of embodiment (100), wherein the monovalent cation is Na⁺.

(102) The product of any of embodiments (81) to (101), wherein the partially desulfated heparin in the preparation is at a concentration of between about 1 mg/mL and about 600 mg/mL.

(103) The product of embodiment (102), wherein the partially desulfated heparin in the preparation is at a concentration of about 50 mg/mL.

(104) The product of embodiment (102), wherein the partially desulfated heparin in the preparation is at a concentration of about 175 mg/mL.

(105) The product of embodiment (102), wherein the partially desulfated heparin in the preparation is at a concentration of about 200 mg/mL.

(106) The product of embodiment (102), wherein the partially desulfated heparin in the preparation is at a concentration of about 400 mg/mL.

(107) The product of any of embodiments (81) to (101), having an osmolality between about 50 mOsm/kg and about 2500 mOsm/kg.

(108) The product of embodiment (107), having an osmolality between about 150 mOsm/kg and about 500 mOsm/kg.

(109) The product of embodiment (108), having an osmolality between about 275 mOsm/kg and about 300 mOsm/kg.

(110) The product of embodiment (109), having an osmolality of about 285 mOsm/kg.

(111) The product of any of embodiments (81) to (101), which is isotonic.

(112) The product of any of embodiments (81) to (101), having a viscosity of less than about 100 cP.

(113) The product of embodiment (112), having a viscosity of less than about 80 cP.

(114) The product of embodiment (113), having a viscosity of less than about 60 cP.

(115) The product of embodiment (114), having a viscosity of less than about 20 cP.

(116) The product of any of embodiments (81) to (115), wherein the partially desulfated heparin in the preparation is of mammalian origin.

(117) The product of embodiment (116), wherein the partially desulfated heparin in the preparation is of bovine origin.

(118) The product of embodiment (116), wherein the partially desulfated heparin in the preparation is of porcine origin.

(119) A preloaded syringe comprising a syringe adapted for subcutaneous administration, and a solution comprising a pharmaceutical composition of any of embodiments (1) to (21), (27) to (43), (46), and (49) to (51).

(120) The preloaded syringe of embodiment (119), wherein the syringe is suitable for self-administration.

(121) The preloaded syringe of embodiment (119) or (120), wherein the syringe is a single use syringe.

(122) The preloaded syringe of any of embodiments (119) to (121), wherein the syringe contains about 0.1 mL to about 5.0 mL of the solution comprising the pharmaceutical composition.

(123) The preloaded syringe of embodiment (122), wherein the syringe contains about 0.5 mL of the solution comprising the pharmaceutical composition.

(124) The preloaded syringe of embodiment (122), wherein the syringe contains about 1.0 mL of the solution comprising the pharmaceutical composition.

(125) The preloaded syringe of embodiment (122), wherein the syringe contains about 2.0 mL of the solution comprising the pharmaceutical composition.

(126) The preloaded syringe of any of embodiments (119) to (125), wherein the partially desulfated heparin is at a concentration of about 1 mg/ml to about 600 mg/mL.

(127) The preloaded syringe of embodiment 1(26), wherein the partially desulfated heparin is at a concentration of about 400 mg/mL.

(128) The preloaded syringe of embodiment (126), wherein the partially desulfated heparin is at a concentration of about 200 mg/mL.

(129) The preloaded syringe of embodiment (126), wherein the partially desulfated heparin is at a concentration of about 175 mg/mL.

(130) The preloaded syringe of any of embodiments (119) to (129), wherein the preloaded syringe comprises 0.2 mg to 800 mg of the partially desulfated heparin.

(131) The preloaded syringe of embodiment (130), wherein the preloaded syringe comprises about 200 mg of the partially desulfated heparin.

(132) An autoinject pen comprising an autoinject pen; and a solution comprising a pharmaceutical composition of any of embodiments (1) to (21), (27) to (43), (46), and (49) to (51).

(133) The autoinject pen of embodiment (132), wherein the amount of injection is predetermined.

(134) The autoinject pen of embodiment (132), wherein the amount of injection is adjusted by the user.

(135) The autoinject pen of any of embodiments (132) to (134), wherein the autoinject pen contains about 0.1 mL to about 5.0 mL of the solution comprising the pharmaceutical composition.

(136) The autoinject pen of embodiment (135), wherein the autoinject pen contains about 0.5 mL of the solution comprising the pharmaceutical composition.

(137) The autoinject pen of embodiment (135), wherein the autoinject pen contains about 1.0 mL of the solution comprising the pharmaceutical composition.

(138) The autoinject pen of embodiment (135), wherein the autoinject pen contains about 5.0 mL of the solution comprising the pharmaceutical composition.

(139) The autoinject pen of any of embodiments (132) to (138), wherein the partially desulfated heparin is at a concentration of about 1 mg/ml to about 600 mg/mL.

(140) The autoinject pen of embodiment (139), wherein the partially desulfated heparin is at a concentration of about 400 mg/mL.

(141) The autoinject pen of embodiment (139), wherein the partially desulfated heparin is at a concentration of about 200 mg/mL.

(142) The autoinject pen of embodiment (139), wherein the partially desulfated heparin is at a concentration of about 175 mg/mL.

(143) The autoinject pen of any of embodiments (132) to (142), comprising 0.2 mg to 800 mg of the partially desulfated heparin.

(144) The autoinject pen of embodiment (143), comprising about 200 mg of the partially desulfated heparin.

(145) A unit dosage form of a partially desulfated heparin, comprising a unit dosage container, and a pharmaceutical composition of any of embodiments (1) to (21), (27) to (43), (46), and (49) to (51).

(146) The unit dosage form of embodiment (145), wherein the container is a vial.

(147) The unit dosage form of embodiment (145), wherein the container is an ampule.

(148) The unit dosage form of embodiment (147), wherein the ampule is suitable for use with a neubulizer.

(149) The unit dosage form of embodiment (145), wherein the container is a syringe.

(150) The unit dosage form of any of embodiments (145) to (149), wherein the unit dosage form is a liquid formulation.

(151) The unit dosage form of any of embodiments (145) to (150), wherein the container contains about 0.1 mL to about 5.0 mL of the pharmaceutical composition.

(152) The unit dosage form of embodiment (151), wherein the container contains about 0.5 mL of the pharmaceutical composition.

(153) The unit dosage form of embodiment (151), wherein the container contains about 1.0 mL of the pharmaceutical composition.

(154) The unit dosage form of embodiment (151), wherein the container contains about 2.0 mL of the pharmaceutical composition.

(155) The unit dosage form of any of embodiments (145) to (154), wherein the partially desulfated heparin is at a concentration of about 10 mg/ml to about 600 mg/mL.

(156) The unit dosage form of embodiment (155), wherein the partially desulfated heparin is at a concentration of about 400 mg/mL.

(157) The unit dosage form of embodiment (155), wherein the partially desulfated heparin is at a concentration of about 200 mg/mL.

(158) The unit dosage form of embodiment (155), wherein the partially desulfated heparin is at a concentration of about 175 mg/mL.

(159) The unit dosage form of embodiment (155), wherein the partially desulfated heparin is at a concentration of about 50 mg/mL.

(160) The unit dosage form of any of embodiments (145) to (159), wherein the container contains 0.2 mg to 800 mg of the partially desulfated heparin.

(161) The unit dosage form of embodiment (160), wherein the container contains about 200 mg of the partially desulfated heparin.

(162) A unit dosage form of a partially desulfated heparin comprising: a unit dosage container, and a pharmaceutical composition of any of embodiments (1) to (21), (36) to (42), (44) or (48) to (51).

(163) The unit dosage form of embodiment (162), wherein the container is a vial.

(164) The unit dosage form of embodiment (162), wherein the container is an ampule.

(165) The unit dosage form of embodiment (162), wherein the container is a metered dose inhaler.

(166) The unit dosage form of any of embodiments (162) to (165), wherein the unit dosage form is a dry formulation.

(167) The unit dosage form of any of embodiments (162) to (166), wherein the container contains 0.2 mg to 800 mg of the partially desulfated heparin.

(168) The unit dosage form of embodiment (167), wherein the container contains about 200 mg of the partially desulfated heparin.

(169) A kit comprising:
i) a preloaded syringe according to any of embodiments (119) to (131); and
ii) instructions for subcutaneous administration.

(170) A kit comprising:
i) an autoinject pen according to any of embodiments (132) to (144); and
ii) instructions for subcutaneous administration.

(171) A kit comprising:
i) an ampule according to embodiment (147); and
ii) instructions for nebulizing administration.

(172) The kit of embodiment (171), further comprising a nebulizer.

(173) A kit comprising:
i) an ampule according to embodiment (164); and
ii) instructions for inhalation administration.

(174) A kit comprising:
i) a metered dose inhaler according to embodiment (166); and
ii) instructions for inhalation administration.

(175) The kit of any of embodiments (169) to (174), further comprising a second therapeutic agent.

(176) The kit of embodiment (175), wherein the second therapeutic agent is capable of treating the same disorder as the partially desulfated heparin.

(177) The kit of embodiment (175), wherein the second therapeutic agent is capable of treating a disorder that is not treatable with the partially desulfated heparin formulation of the kit.

(178) The kit of embodiment (175), wherein the second therapeutic agent is for the treatment or prevention of acute radiation syndrome.

(179) The kit of embodiments (169) to (174), further comprising a separate treatment for a radiation induced harm other than thrombocytopenia.

(180) The kit of embodiment (179), wherein the separate treatment is iodine.

(181) The kit of embodiment (175), wherein the second therapeutic agent is effective in the treatment of cancer.

(182) The kit of embodiment (175), wherein the second therapeutic agent in preventing damage caused by ischemia reperfusion.

(183) The kit of embodiment (175), wherein the second therapeutic agent is effective for the treatment of asthma, chronic bronchitis, emphysema, acute respiratory distress syndrome, cystic fibrosis or bacterial or viral infections.

(184) A method for treating acute radiation syndrome, comprising administering subcutaneously to a subject exposed to ionizing radiation a partially desulfated heparin associated with a multivalent cation in an amount effective to treat a complication of acute radiation syndrome.

(185) A method for treating hematologic injury, comprising administering subcutaneously to a subject in need of treatment for a hematologic injury a partially desulfated heparin associated with a multivalent cation in an amount effective to treat the hematologic injury.

(186) A method for preventing hematologic injury, comprising administering subcutaneously to a subject at risk of a hematologic injury a partially desulfated heparin associated with a multivalent cation in an amount effective to prevent the hematologic injury.

(187) A method for treating thrombocytopenia, comprising administering subcutaneously to a subject in need of treatment thereof a partially desulfated heparin associated with a multivalent cation in an amount effective to treat thrombocytopenia.

(188) The method of embodiment (187), wherein the thrombocytopenia is heparin-induced thrombocytopenia.

(189) The method of embodiment (187), wherein the thrombocytopenia is radiation-induced thrombocytopenia.

(190) The method of embodiment (187), wherein the thrombocytopenia is associated with reduced production of platelets.

(191) A method for treating or preventing ischemia reperfusion injury, comprising administering subcutaneously to a subject in need of treatment thereof or at risk therefor a partially desulfated heparin associated with a multivalent cation in an amount effective to treat or prevent ischemia reperfusion injury.

(192) A method for treating chronic bronchitis, comprising administering to a subject in need of treatment for chronic bronchitis a partially desulfated heparin associated with a multivalent cation in an amount effective to treat chronic bronchitis.

(193) A method for treating asthma, comprising administering to a subject in need of treatment for asthma a partially desulfated heparin associated with a multivalent cation in an amount effective to treat asthma, wherein the administration is by inhalation.

(194) A method for treating emphysema, comprising administering to a subject in need of treatment for emphysema a partially desulfated heparin associated with a multivalent cation in an amount effective to treat emphysema, wherein the administration is by inhalation.

(195) A method for treating acute respiratory distress syndrome, comprising administering to a subject in need of treatment for acute respiratory distress syndrome a partially desulfated heparin associated with a multivalent cation in an amount effective to treat acute respiratory distress syndrome, wherein the administration is by inhalation.

(196) A method for treating cystic fibrosis, comprising administering to a subject in need of treatment for cystic fibrosis a partially desulfated heparin associated with a multivalent cation in an amount effective to treat cystic fibrosis, wherein the administration is by inhalation.

(197) A method for treating bacterial or viral infections of the lung, comprising administering to a subject in need of treatment for a bacterial or viral infection of the lung a partially desulfated heparin associated with a multivalent cation in an amount effective to treat a bacterial or viral infection of the lung, wherein the administration is by inhalation.

(198) A method for treating rhinitis in a subject, comprising administering to a subject in need of treatment for rhinitis a partially desulfated heparin associated with a multivalent cation in an amount of effective to treat rhinitis.

(199) A method for preventing rhinitis in a subject, comprising administering to a subject at risk of rhinitis a partially desulfated heparin associated with a multivalent cation in an amount effective to prevent rhinitis.

(200) A method for treating sinusitis in a subject, comprising t administering to a subject in need of treatment for sinusitis a partially desulfated heparin associated with a multivalent cation in an amount effective to treat sinusitis.

(201) A method for preventing sinusitis in a subject, comprising administering to a subject at risk of sinusitis a partially desulfated heparin associated with a multivalent cation in an amount effective to prevent sinusitis.

(202) The method according to any of embodiments (184) to (201), wherein the partially desulfated heparin is at least about 50% desulfated at the 2-O position.

(203) The method according to embodiment (202), wherein the partially desulfated heparin is at least about 99% desulfated at the 2-O position.

(204) The method according to any of embodiments (184) to (201), wherein the partially desulfated heparin is at least about 50% desulfated at the 3-O position.

(205) The method according to embodiment (204), wherein the partially desulfated heparin is at least about 99% desulfated at the 3-O position.

(206) The method according to any of embodiments (184) to (201), wherein the partially desulfated heparin is at least about 50% desulfated at each of the 2-O and 3-O positions.

(207) The method according to embodiment (206), wherein the partially desulfated heparin is at least about 99% desulfated at each of the 2-O and 3-O positions.

(208) The method according to any of embodiments (184) to (201), wherein the partially desulfated heparin is ODSH.

(209) The method according to any of embodiments (184) to (208), wherein the partially desulfated heparin comprises substantially N-sulfated and 6-O sulfated D-glucosamine and substantially intact carboxylates of α-L-iduronic acid.

(210) The method according to any of embodiments (184) to (209), wherein the partially desulfated heparin has an average molecular weight of greater than about 2 kDa.

(211) The method according to embodiment (210), wherein the partially desulfated heparin has an average molecular weight of about 8 kDa to about 15 kDa.

(212) The method according to embodiment (211), wherein the partially desulfated heparin has an average molecular weight of about 11 kDa to about 13 kDa.

(213) The method according to any of embodiments (184) to (212), wherein the multivalent cation is a divalent cation.

(214) The method according to embodiment (213), wherein the divalent cation is Mg²⁺.

(215) The method according to embodiment (214), wherein the divalent cation is Ca²⁺.

(216) The method according to any of embodiments (184) to (215), wherein the multivalent cation is at a concentration of at least about equal to the concentration of a monovalent cation.

(217) The method according to embodiment (216), wherein the multivalent cation is at a concentration of at least about twice the concentration of the monovalent cation.

(218) The method according to embodiment (217), wherein the multivalent cation is at a concentration of at least about ten times the concentration of the monovalent cation.

(219) The method according to embodiment (218), wherein the partially desulfated heparin is substantially free of the monovalent cation.

(220) The method according to any of embodiments (216) to (219), wherein the monovalent cation is Na⁺, Li⁺, K⁺, or combinations thereof.

(221) The method according to embodiment (220), wherein the monovalent cation is Na⁺.

(222) The method according to any of embodiments (184) to (221), wherein the partially desulfated heparin is administered at a dose of between about 0.1 mg/kg and about 50 mg/kg.

(223) The method according to embodiment (222), wherein the dose of the partially desulfated heparin is about 25 mg/kg.

(224) The method according to embodiment (222), wherein the dose of the partially desulfated heparin is about 8 mg/kg.

(225) The method according to embodiment (222), wherein the dose of the partially desulfated heparin is about 2 mg/kg.

(226) The method according to embodiment (222), wherein the dose of the partially desulfated heparin is about 1 mg/kg.

(227) The method according to any of embodiments (184) to (221), wherein the partially desulfated heparin is administered as a fixed total body dose between about 0.5 mg and about 5000 mg.

(228) The method according to embodiment (227), wherein the fixed total body dose of the partially desulfated heparin is about 500 mg.

(229) The method according to embodiment (227), wherein the fixed total body dose of the partially desulfated heparin is about 200 mg.

(230) The method according to embodiment (227), wherein the fixed total body dose of the partially desulfated heparin is about 100 mg.

(231) The method according to embodiment (227), wherein the fixed total body dose of the partially desulfated heparin is about 50 mg.

(232) The method according to any of embodiments (184) to (231), wherein the partially desulfated heparin is of mammalian origin.

(233) The method according to embodiment (232), wherein the partially desulfated heparin is of bovine origin.

(234) The method according to embodiment (232), wherein the partially desulfated heparin is of porcine origin.

As will be apparent to the skilled artisan, the foregoing provides various combinations of embodiments, and each embodiment which can be made by the combinations constitutes part of the disclosure.

### 5. EXAMPLES

Various features of the invention will be shown through the following non-limiting examples.

### Example 1

### Osmolality of Na⁺-ODSH Formulations

In order to determine the limits of Na⁺-ODSH formulations suitable for human use, the osmolality of ascending concentrations of Na⁺-ODSH was measured.

Methods: pH was measured using a pH electrode according to methods known in the art. Osmolality was measured using the freezing-point depression method as is known in the art. Viscosity was measured using a Brookfield RVDV-E viscometer at 25° C. at a spindle speed of 100 rpm according to manufacturer's instructions.

Results: pH, osmolality and viscosity of Na⁺-ODSH concentrations ranging from 21 mg/mL to 600 mg/mL are listed in Table 1. For reference, normal human plasma osmolality ranges from 270 mOsm/kg to 295 mOsm/kg. Osmolality substantially above this range is painful to administer to a subject.

TABLE 1
Osmolality of Na⁺-ODSH Solutions

| ODSH | | Osmolality | Viscosity |
|---|---|---|---|
| (mg/mL) | pH | (mOsm/kg) | (cP) |
| 21 | 5.83 | 52 | NA |
| 51 | 5.47 | 129 | NA |
| 100 | 5.23 | 274 | NA |
| 200 | 4.99 | 634 | NA |
| 300 | 4.95 | 1086 | 21 |
| 401 | 4.89 | 1506 | 81 |
| | | (estimated) | |
| 600 | 4.76 | 2531 | 708 |

### Example 2

### Cation Substitution

Multiple cations were selected to test formulations for desulfated heparin. ODSH was used as the test desulfated heparin. Zn²⁺, Ca²⁺, Mg²⁺, Fe²⁺ and Lysine were tested for making ODSH formulations.

Methods: A 200 mg/mL Na⁺-ODSH solution was prepared in a concentrated solution of divalent cation with 20 mM Tris. The solution was filtered through a 0.2 μm PVDF filter. After passing through the filter, excess free cations were removed using a desalting column. The solution was passed through a Spehadex-G25 column aided by centrifugation at 2000 rpm for 10 minutes.

Osmolality was measured using the freezing-point depression method as is known in the art. ODSH concentration was measured by ultraviolet UV spectrophotometry at 314 nm against a standard curve from the stock Na⁺-ODSH.

Results: Use of multivalent cations successfully reduced the osmolality of ODSH solutions. Particular concentration and osmolality values are given below in Table 2. Ca²⁺ and Mg²⁺ each generated solutions of acceptable osmolality at concentrations higher than Na⁺-ODSH.

TABLE 2
Multivalent Cation ODSH Formulations

| | Cation | ODSH | Osmolality |
|---|---|---|---|
| Complexing Agent | Concentration | (mg/mL) | (mOsm/kg) |
| Zinc Chloride | 1M | 226 | 693 |
| Calcium Chloride | 2M | 172 | 274 |
| Magnesium Chloride | 2M | 156 | 318 |
| Ferric (Fe³⁺) Chloride | 2M | ND | 643 |
| Lysine | 2M | 44 | 150 |

### Example 3

### Cation Substitution for Higher Concentrations of Ca²⁺-ODSH

Higher concentrations of ODSH could not effectively have monovalent (Na⁻) cations removed by use of the desalting column due to the viscosity of the liquid. Therefore, desalting was also performed using dialysis.

Methods: A range from 200 mg/mL to 800 mg/L of Na⁺-ODSH solutions was prepared in a concentrated solution of 2 M Calcium Chloride. The solution was filtered through a 0.2 μm PVDF filter. After passing through the filter, excess free cations were removed by dialysis. Dialysis was performed overnight against water using a membrane with a 2000 Da molecular weight cutoff. The dialysate was then concentrated by lyophilizing and reconstituting in 20% of the initial volume with 20 mM Tris.

Osmolality was measured using the freezing-point depression method as is known in the art. Viscosity was measured using a Brookfield RVDV-E viscometer at 25° C. at a spindle speed of 100 rpm according to manufacturer's instructions. ODSH concentration was measured by ultraviolet UV spectrophotometry at 314 nm and by activity in a Human Leukocyte Elastase (HLE) inhibition assay. Methods for measuring HLE inhibition are known in the art. See, e.g., Fryer et al., J. Pharmacol. Exp. Ther. 282:208-219 (1997). The relationship between concentration and HLE inhibition in the standard curve was not linear, following the equation:

% Enzyme inhibition=94(1−e^{0.43(ODSH mg/mL)})

Results: Use of multivalent cations successfully reduced the osmolality of ODSH solutions.

Particular values for two separate preparations are given below in Table 3.

TABLE 3
Ca²⁺-ODSH Formulations
Preparation 1

| | | ODSH | |
|---|---|---|---|
| | Osmolality | (mg/mL) | |
| Sample | (mOsm/kg) | UV | Activity |
| Pre-dialyzed | Did not freeze | 184.0 | 135.0 |
| Dialyzed | 80 | 85.2 | 73.4 |
| Lyophilized and | 197 | 362.5 | 289.1 |
| Reconstituted | (1:1 dilution) | | |

Preparation 2

| | | ODSH | |
|---|---|---|---|
| | Osmolality | (mg/mL) | |
| Sample | (mOsm/kg) | UV | Activity |
| Pre-dialyzed | Did not freeze | 174.8 | 125.4 |
| Dialyzed | 89 | 75.6 | 61.2 |
| Lyophilized and | 221 | 320.3 | 295.4 |
| Reconstituted | (1:1 dilution) | | |

Ca²⁺-ODSH was successfully made at concentrations greater than 300 mg/mL. Based on the osmolality of a 1:1 dilution (˜200 mOsm/kg), the undiluted sample osmolality would be ˜400 mOsm/kg. 400 mOsm/kg is within the acceptable range for an injectable formulation. The viscosity of the reconstituted samples was 60 cP.

### Example 4

### Syringibility and Filterability of High Concentration Ca²⁺-ODSH

In order to determine the feasibility of using high concentration Ca²⁺-ODSH for pharmaceutical compositions, the ability of such a formulation was assessed for the ease with which it can be filtered or pushed through a syringe.

Methods: Ca²⁺-ODSH was prepared at ˜230 mg/mL and diluted to the concentrations in Tables 4 and 5. 1 mL polypropylene disposable syringes with either a 23 gauge or 25 gauge needle were filled with 1 mL of the solution. For filterability experiments, a 13 mm diameter, 0.2 μm PVDF filter was used with a 23 gauge needle. A 500 g weight was placed on top of the plunger, and the time for the entire contents of the syringe to flow through the needles was measured. In filterability experiments, the weight and activity of the solution were measured before and after filtration. Activity was assessed using the HLE inhibition assay and reported as the concentration of ODSH as described above in Example 3.

Results: Results from duplicate syringibility experiments are shown in Table 4. Syringibility was better with a 23 gauge needle compared to a 25 gauge needle. Syringibility was also better with reduced concentration. The differences in syringibility between the 23 gauge and 25 gauge needle decreased with lower concentrations.

TABLE 4
Syringibility of High Concentration ODSH Formulations

| | 25 gauge | | 23 gauge | |
|---|---|---|---|---|
| Solution | Run 1 | Run 2 | Run 1 | Run 2 |
| Water | 0:03 | 0:03 | 0:02 | 0:03 |
| 230 mg/mL ODSH | 3:25 | 3:29 | 2:21 | 2:29 |
| 209 mg/mL ODSH | 1:40 | 1:42 | 1:00 | 1:01 |
| 192 mg/mL ODSH | 1:13 | 1:08 | 0:52 | 0:52 |
| 177 mg/mL ODSH | 0:52 | 0:49 | 0:36 | 0:33 |

Results from duplicate filterability experiments are shown in Table 5. For the 230 mg/mL solution, only 0.8 mL of solution flowed through under the 500 g weight. The remainder was pushed through by hand. The percentage of volume recovered did not vary with concentration, suggesting that the hold-up volume of the syringe is responsible for the loss. The estimated hold-up volume is about 180 μL. Greater than 90% of the activity was recovered at all concentrations indicating that filtration does not have a substantial effect on activity.

TABLE 5
Filterability of High Concentration ODSH Formulations

| | | Recovery after filtration | | | |
|---|---|---|---|---|---|
| | | % Volume | Activity (mg/mL) | | |
| Solution | Time | Recovered | Pre-filtered | Filtered | % Recovery |
| 230 mg/mL ODSH | 74:33 | 82.4 | 230.5 | 207.9 | 90.2 |
| 209 mg/mL ODSH | 33:31 | 82.3 | 213.5 | 205.5 | 96.3 |
| 192 mg/mL ODSH | 21:20 | 82.8 | 202.6 | 209.9 | 103.6 |

### 6. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 7. EQUIVALENTS

The present disclosure provides, inter alia, compositions of multivalent cation associated heparins. The present disclosure also provides processes for making the multivalent cation-associated heparins, products of those processes, unit dosage forms containing the compositions, and methods of using the compositions. While various specific embodiments have been illustrated and described, the above specification is not restrictive. It will be appreciated that various changes can be made without departing from the spirit and scope of the invention(s). Many variations will become apparent to those skilled in the art upon review of this specification.

What is claimed is:

1. A pharmaceutical composition comprising a partially desulfated heparin, a multivalent cation, and a pharmaceutically acceptable carrier.

2. The pharmaceutical composition of claim 1, wherein the multivalent cation is a divalent cation.

3. The pharmaceutical composition of claim 2, wherein the divalent cation is Mg²⁺.

4. The pharmaceutical composition of claim 2, wherein the divalent cation is Ca²⁺.

5. The pharmaceutical composition of any of claims 1 to 4, wherein the multivalent cation is at a concentration of at least about equal to the concentration of a monovalent cation.

6. The pharmaceutical composition of claim 5, wherein the multivalent cation is at a concentration of at least about twice the concentration of the monovalent cation.

7. The pharmaceutical composition of claim 6, wherein the multivalent cation is at a concentration of at least about ten times the concentration of the monovalent cation.

8. The pharmaceutical composition of claim 7, wherein the pharmaceutical composition is substantially free of the monovalent cation.

9. The pharmaceutical composition of any of claims 5 to 8, wherein the monovalent cation is Na⁺, Li⁺, K⁺, or combinations thereof.

10. The pharmaceutical composition of any one of claims 1 to 9, wherein the partially desulfated heparin is at least about 50% desulfated at the 2-O position.

11. The pharmaceutical composition of claim 10, wherein the partially desulfated heparin is at least about 99% desulfated at the 2-O position.

12. The pharmaceutical composition of any one of claims 1 to 9, wherein the partially desulfated heparin is at least about 50% desulfated at the 3-O position.

13. The pharmaceutical composition of claim 12, wherein the partially desulfated heparin is at least about 99% desulfated at the 3-O position.

14. The pharmaceutical composition of any one of claims 1 to 9, wherein the partially desulfated heparin is at least about 50% desulfated at each of the 2-O and 3-O positions.

15. The pharmaceutical composition of claim 14, wherein the partially desulfated heparin is at least about 99% desulfated at each of the 2-O and 3-O positions.

16. The pharmaceutical composition of claim 1, wherein the partially desulfated heparin is ODSH.

17. The pharmaceutical composition of any of claims 1 to 16, wherein the partially desulfated heparin comprises substantially N-sulfated and 6-O sulfated D-glucosamine and substantially intact carboxylates of α-L-iduronic acid.

18. The pharmaceutical composition of any of claims 1 to 17, wherein the partially desulfated heparin has an average molecular weight of greater than about 2 kDa.

19. The pharmaceutical composition of claim 18, wherein the partially desulfated heparin has an average molecular weight of about 8 kDa to about 15 kDa.

20. The pharmaceutical composition of claim 19, wherein the partially desulfated heparin has an average molecular weight of about 11 kDa to about 13 kDa.

21. A method for preparing a partially desulfated heparin associated with a multivalent cation, comprising the steps of:
(i) adjusting the pH of a solution of unfractionated heparin to at least 13.0;
(ii) drying the alkaline solution of heparin of step (i);
(iii) reconstituting the dried heparin as a solution with the multivalent cation; and,
(iv) removing excess cations.

22. The product generated by the method of claim 21.
